(19) United States
(12) Patent Application Publication
(10) Pub. No.: US 2022/0016205 A1
(43) Pub. Date: Jan. 20, 2022

(54) METHODS OF OVERCOMING RESISTANCE TO IMMUNE CHECKPOINT INHIBITORS

(71) Applicant: Board of Regents, The University of Texas System, Austin, TX (US)

(72) Inventors: James WELSH, Houston, TX (US); Maria Angelica CORTEZ, Houston, TX (US)

(73) Assignee: Board of Regents, The University of Texas System, Austin, TX (US)

(21) Appl. No.: 17/295,747

(22) PCT Filed: Nov. 21, 2019
(86) PCT No.: PCT/US2019/062654

§ 371 Date: May 20, 2021

Related U.S. Application Data
(60) Provisional application No. 62/770,319, filed on Nov. 21, 2018.

(51) Int. Cl. A61K 38/17; A61K 39/395; A61K 45/06; G01N 33/574; C07K 16/28; A61P 35/00

(52) U.S. Cl. CPC A61K 38/1709; A61K 39/3955; A61K 45/06; G01N 2333/51; C07K 16/2818; A61P 35/00; G01N 33/57484

(57) ABSTRACT

Provided herein are methods of using BMP7 levels as a marker for the selection of patients, such as non-small cell lung cancer patients, who will clinically respond to combination therapy comprising a BMP7 inhibitor and an immune checkpoint therapy, such as an anti-PD1 therapy and/or an anti-CTLA-4 therapy. Also provided are methods of treating the selected patients with a combination of a BMP7 inhibitor and an immune checkpoint therapy.

### REFERENCE TO RELATED APPLICATIONS

The present application claims the priority benefit of U.S. provisional application No. 62/770,319, filed Nov. 21, 2018, the entire contents of which is incorporated herein by reference.

### BACKGROUND

### 1. Field

The present invention relates generally to the fields of cancer biology and immunotherapy. More particularly, it concerns methods for selecting patients for treatment with a combination of a BMP7 inhibitor and an immune checkpoint therapy as well as treating patients so selected.

### 2. Description of Related Art

Reactivation of the immune system from the use of checkpoint inhibitors is one of the most profound advances in cancer therapy. Through the blockage of CTLA-4 and PD1/PD-L1 T cells can be activated to attack a patient cancer from the inside. This approach has been validated through the recent FDA approval of these agents for melanoma, and they are now being rapidly expanded into most other solid tumors. Despite the significant promise of these agents the majority of patients do not respond, and resistance can also develop. Strategies that can improve their efficacy and or address checkpoint resistance are needed.

### SUMMARY

Provided herein are therapeutic targets that can favorably influence the tumor microenvironment in a manner that improves T cell function and tumor penetration. For example, BMP7 inhibition with neutralizing antibodies, inhibitory nucleic acids, and/or small molecules may be used in combination with immune checkpoint inhibitors to overcome immune checkpoint blockade resistance.

In one embodiment, provided herein are methods for the treatment of a cancer in a patient, the methods comprising administering to the patient a combined effective amount of a BMP7 inhibitor and an immune checkpoint inhibitor. In some aspects, the patient has previously failed to respond to the administration of an immune checkpoint inhibitor, such as, for example, an anti-PD1, an anti-PD-L1 therapy, and/or an anti-CTLA-4 therapy.

In some aspects, the patient has an increased level of BMP7 relative to a BMP7 level in a references sample. The patient's increased level of BMP7 may be detectable within the cancer itself or within the patient's serum. In some aspects, the patient's cancer expresses an increased level of beta-catenin, Sox2, and/or PARP1 relative to a beta-catenin, Sox2, and/or PARP1 level in a reference sample. In some aspects, the patient's cancer expresses a decreased level of CDKN2A, p38α, PTEN, PD-L1, YAP1_pS127, and/or granzyme B relative to a CDKN2A, p38α, PTEN, PD-L1, YAP1_pS127, and/or granzyme B level in a reference sample. In some aspects, a tumor infiltrating lymphocyte in the patient's cancer expresses a decreased level of IL-1α, TNF-α, IFN-γ, and/or IL-2 relative to an IL-1α, TNF-α, IFN-γ, and/or IL-2 level in a reference sample. A reference sample may be sourced from healthy or non-cancerous tissue from the patient or from a healthy subject.

In some aspects, the BMP7 inhibitor comprises a BMP7 antagonist protein, a BMP7 neutralizing antibody, an inhibitory nucleic acid targeting BMP7 mRNA, or a BMP7 antagonist small molecule. In certain aspects, the BMP7 antagonist protein is follistatin, noggin, or uterine sensitization-associated gene-1 (USAG-1). In some aspects, the BMP7 antagonist protein may be PEGylated. In certain aspects, the BMP7 antagonist small molecule is K02288. In certain aspects, the inhibitory nucleic acid targeting BMP7 mRNA comprises a BMP7 shRNA or siRNA. In some aspects, the BMP7 inhibitor is comprised in a lipid nanoparticle, such as, for example, an exosome. In some aspects, the BMP7 inhibitor is comprised in a nanoshuttle for controlled intratumoral delivery.

In some aspects, the immune checkpoint inhibitor comprises one or more of an anti-PD1 therapy, an anti-PD-L1 therapy, and an anti-CTLA-4 therapy. In some aspects, the anti-PD1 therapy comprises nivolumab, pembrolizumab, pidilizumab, AMP-223, AMP-514, cemiplimab, or PDR-001. In some aspects, the anti-PD-L1 therapy comprises atezolizumab, avelumab, durvalumab, BMS-036559, or CK-301. In some aspects, the anti-CTLA-4 therapy comprises ipilimumab or tremelimumab.

In some aspects, the methods further comprise administering a further anti-cancer therapy to the patient. In certain aspects, the second anti-cancer therapy is a surgical therapy, chemotherapy, radiation therapy, cryotherapy, hormonal therapy, toxin therapy, immunotherapy, or cytokine therapy. In certain aspects, the surgical therapy comprises a pneumonectomy, a lobectomy, a segmentectomy, a wedge resection, or a sleeve resection. In certain aspects, the radiation therapy comprises external beam radiation therapy or brachytherapy. In certain aspects, the chemotherapy comprises the administration of one or more agents selected from the group consisting of cisplatin, carboplatin, paclitaxel, albumin-bound paclitaxel, docetaxel, gemcitabine, vinorelbine, irinotecan, etoposide, vinblastine, and pemetrexed. In certain aspects, the further anti-cancer therapy comprises erlotinib, afatinib, gefitinib, osimertinib, or dacomitinib if the patient's cancer expresses an increased level of EGFR relative to a reference level. In certain aspects, the further anti-cancer therapy comprises crizotinib, ceritinib, alectinib, brigatinib, or lorlatinib if the patient's cancer has an ALK gene rearrangement. In certain aspects, the further anti-cancer therapy comprises dabrafenib or trametinib if the patient's cancer expresses an altered BRAF protein.

In some aspects, the cancer is a lung cancer or a breast cancer. In certain aspects, the lung cancer is a non-small cell lung cancer. In certain aspects, the breast cancer is a triple-negative breast cancer.

In some aspects, the patient has previously undergone at least one round of anti-cancer therapy. In some aspects, the patient is a human. In some aspects, the methods further comprise reporting the BMP7, beta-catenin, Sox2, PARP1, CDKN2A, p38α, PTEN, PD-L1, YAP1_pS127, granzyme B, IL-1α, TNF-α, IFN-γ, and/or IL-2 expression level. In certain aspects, the reporting comprises preparing a written or electronic report. In certain aspects, the methods further comprise providing the report to the subject, a doctor, a hospital, or an insurance company.

In one embodiment, methods are provided for selecting a patient having a cancer for treatment with a combined effective amount of a BMP7 inhibitor and an immune checkpoint inhibitor, the methods comprising (a) determining whether (i) the patient's cancer has an increased level of BMP7 relative to a BMP7 level in a reference sample, (ii) the patient's serum comprises an increased level of BMP7 relative to a BMP7 level in a reference sample, (iii) the patient's cancer expresses an increased level of beta-catenin, Sox2, and/or PARP1 relative to a beta-catenin, Sox2, and/or PARP1 level in a reference sample, (iv) the patient's cancer expresses a decreased level of CDKN2A, p38α, PTEN, PD-L1, YAP1_pS127, and/or granzyme B relative to a CDKN2A, p38α, PTEN, PD-L1, YAP1_pS127, and/or granzyme B level in a reference sample, or (v) a tumor infiltrating lymphocyte in the patient's cancer expresses a decreased level of IL-1α, TNF-α, IFN-γ, and/or IL-2 relative to an IL-1α, TNF-α, IFN-γ, and/or IL-2 level in a reference sample, and (b) selecting the patient for treatment if the patient's cancer has an increased level of BMP7 relative to a BMP7 level in a reference sample.

In one embodiment, methods are provided for selecting a patient having a cancer for treatment with a combined effective amount of a BMP7 inhibitor and an immune checkpoint inhibitor, the methods comprising (a) determining whether the patient's cancer has an increased level of BMP7 relative to a BMP7 level in a reference sample, and (b) selecting the patient for treatment if the patient's cancer has an increased level of BMP7 relative to a BMP7 level in a reference sample.

In some aspects, the methods further comprise administering a combined effective amount of a BMP7 inhibitor and an immune checkpoint inhibitor to the selected patient. In some aspects, the methods further comprise selecting the patient for treatment if the patient has previously failed to respond to the administration of an immune checkpoint inhibitor. In certain aspects, the immune checkpoint inhibitor comprises an anti-PD1 and/or anti-PD-L1 therapy.

In some aspects, the methods further comprise selecting the patient for treatment if the patient's serum comprises an increased level of BMP7 relative to a BMP7 level in a reference sample. In some aspects, the methods further comprise selecting the patient for treatment if the patient's cancer expresses an increased level of beta-catenin, Sox2, and/or PARP1 relative to a beta-catenin, Sox2, and/or PARP1 level in a reference sample. In some aspects, the methods further comprise selecting the patient for treatment if the patient's cancer expresses a decreased level of CDKN2A, p38α, PTEN, PD-L1, YAP1_pS127, and/or granzyme B relative to a CDKN2A, p38α, PTEN, PD-L1, YAP1_pS127, and/or granzyme B level in a reference sample. In some aspects, the methods further comprise selecting the patient for treatment if a tumor infiltrating lymphocyte in the patient's cancer expresses a decreased level of IL-1α, TNF-α, IFN-γ, and/or IL-2 relative to an IL-1α, TNF-α, IFN-γ, and/or IL-2 level in a reference sample. A reference sample may be sourced from healthy or non-cancerous tissue from the patient or from a healthy subject.

In some aspects, the BMP7 inhibitor comprises a BMP7 antagonist protein, a BMP7 neutralizing antibody, an inhibitory nucleic acid targeting BMP7 mRNA, or a BMP7 antagonist small molecule. In certain aspects, the BMP7 antagonist protein is follistatin, noggin, or uterine sensitization-associated gene-1 (USAG-1). In some aspects, the BMP7 antagonist protein may be PEGylated. In some aspects, the BMP7 antagonist small molecule is K02288. In some aspects, the inhibitory nucleic acid targeting BMP7 mRNA comprises a BMP7 shRNA or siRNA. In certain aspects, the BMP7 inhibitor is comprised in a lipid nanoparticle, such as, for example, an exosome. In certain aspects, the BMP7 inhibitor is comprised in a nanoshuttle for controlled intratumoral delivery.

In some aspects, the immune checkpoint inhibitor comprises one or more of an anti-PD1 therapy, an anti-PD-L1 therapy, and an anti-CTLA-4 therapy. In some aspects, the anti-PD1 therapy comprises nivolumab, pembrolizumab, pidilizumab, AMP-223, AMP-514, cemiplimab, or PDR-001. In some aspects, the anti-PD-L1 therapy comprises atezolizumab, avelumab, durvalumab, BMS-036559, or CK-301. In some aspects, the anti-CTLA-4 therapy comprises ipilimumab or tremelimumab.

In some aspects, the methods further comprise administering a further anti-cancer therapy to the patient. In certain aspects, the second anti-cancer therapy is a surgical therapy, chemotherapy, radiation therapy, cryotherapy, hormonal therapy, toxin therapy, immunotherapy, or cytokine therapy. In certain aspects, the surgical therapy comprises a pneumonectomy, a lobectomy, a segmentectomy, a wedge resection, or a sleeve resection. In certain aspects, the radiation therapy comprises external beam radiation therapy or brachytherapy. In certain aspects, the chemotherapy comprises the administration of one or more agents selected from the group consisting of cisplatin, carboplatin, paclitaxel, albumin-bound paclitaxel, docetaxel, gemcitabine, vinorelbine, irinotecan, etoposide, vinblastine, and pemetrexed. In certain aspects, the further anti-cancer therapy comprises erlotinib, afatinib, gefitinib, osimertinib, or dacomitinib if the patient's cancer expresses an increased level of EGFR relative to a reference level. In certain aspects, the further anti-cancer therapy comprises crizotinib, ceritinib, alectinib, brigatinib, or lorlatinib if the patient's cancer has an ALK gene rearrangement. In certain aspects, the further anti-cancer therapy comprises dabrafenib or trametinib if the patient's cancer expresses an altered BRAF protein.

In some aspects, the cancer is a lung cancer or a breast cancer. In certain aspects, the lung cancer is a non-small cell lung cancer. In certain aspects, the breast cancer is a triple-negative breast cancer. In some aspects, the patient has previously undergone at least one round of anti-cancer therapy. In some aspects, the patient is a human.

In some aspects, the methods further comprise reporting the BMP7 level in the patient's cancer. In certain aspects, the reporting comprises preparing a written or electronic report. In certain aspects, the methods further comprise providing the report to the subject, a doctor, a hospital, or an insurance company.

In one embodiment, provided herein are pharmaceutical formulations comprising a BMP7 inhibitor and an immune checkpoint inhibitor. In some aspects, the BMP7 inhibitor comprises a BMP7 antagonist protein, a BMP7 neutralizing antibody, an inhibitory nucleic acid targeting BMP7 mRNA, or a BMP7 antagonist small molecule. In certain aspects, the BMP7 antagonist protein is follistatin, noggin, or uterine sensitization-associated gene-1 (USAG-1). In some aspects, the BMP7 antagonist protein may be PEGylated. In certain aspects, the BMP7 antagonist small molecule is K02288. In certain aspects, the inhibitory nucleic acid targeting BMP7 mRNA comprises a BMP7 shRNA or siRNA. In some aspects, the BMP7 inhibitor is comprised in a lipid nanoparticle, such as, for example, an exosome. In some aspects, the BMP7 inhibitor is comprised in a nanoshuttle for controlled intratumoral delivery. In some aspects, the immune checkpoint inhibitor comprises one or more of an anti-PD1 therapy, an anti-PD-L1 therapy, and an anti-CTLA-4 therapy. In certain aspects, the anti-PD1 therapy comprises nivolumab, pembrolizumab, pidilizumab, AMP-223, AMP-514, cemiplimab, or PDR-001. In certain aspects, the anti-PD-L1 therapy comprises atezolizumab, avelumab, durvalumab, BMS-036559, or CK-301. In certain aspects, the anti-CTLA-4 therapy comprises ipilimumab or tremelimumab.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, the variation that exists among the study subjects, or a value that is within 10% of a stated value.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1D. BMP7 is upregulated in tumors resistant to immunotherapies. FIG. 1A provides pyrosequencing methylation assay results with specific primers for BMP7 CpG. FIG. 1B provides ELISA analysis of BMP7 levels in serum of mice bearing 344SQR (n=3) or 344SQP (n=3) tumors treated with anti-PD1. FIG. 1C provides qPCR analysis of BMP7 expression in 344SQP (n=3) and 344SQR (n=3) tumors treated with anti-PD1. ACTB expression was used as a housekeeping gene for qPCR analysis. FIG. 1D provides representative images of IHC stains of BMP7 expression in formalin-fixed paraffin-embedded tissue sections from 344SQP and 344SQR tumors treated with anti-PD1. Scale bar, 100 μm (40× magnification).

FIGS. 2A-2K. BMP7 modulates p38a in anti-PD1-resistant tumors and tumor-infiltrating lymphocytes. FIG. 2A provides reverse phase protein array (RPPA) results on expression levels and activation status of 243 proteins in 344SQP (n=3) and 344SQR (n=3) tumors treated with anti-PD1. Normalized data were first log 2-transformed (log 2(x+1)). Proteins expressed at different levels between groups were identified by a P value of <0.05 obtained from LIMMA's moderated t-statistic. FIG. 2B provides representative images of immunohistochemical stains for p38α, SMAD1/5/9 phosphorylation, and SMAD1 in formalin-fixed paraffin-embedded tissue sections from 344SQP and 344SQR tumors treated with anti-PD1. Scale bar, 100 μm (40× magnification). FIG. 2C provides analysis of the level of BMP7 expression in 344SQR stable cell lines overexpressing shRNAs against BMP7 versus 344SQR cells lines overexpressing control shRNAs. FIG. 2D provides Western blot analysis of the level of BMP7 expression in 344SQR stable cell lines overexpressing shRNAs against BMP7 versus 344SQR cells lines overexpressing control shRNAs. FIG. 2E provides representative images of immunohistochemical stains for p38α, SMAD1/5/9 phosphorylation, and SMAD1 in formalin-fixed paraffin-embedded tissue sections from BMP7 knockdown tumors treated with anti-PD1 compared with control (scale bar, 100 μm) (40× magnification). FIG. 2F provides qPCR analysis of p38a and BMP7 expression in BMP7 knockout tumors treated with anti-PD1. FIG. 2G provides expression analyses for Beta Catenin, PARP1, SOX2, and ETS1 in control and BMP7-knockout 344SQR tumors treated with anti-PD1. FIG. 2H provides Nanostring immune panel results for 770 genes in tumor-infiltrating lymphocytes (TILs) collected from 344SQP (n=2) and 344SQR (n=3) tumors treated with anti-PD1. Genes expressed at different levels between groups were identified by a P value of <0.05 obtained from LIMMA's moderated t statistic. FIG. 2I provides ELISA analysis of plasma levels of cytokines and chemokines regulated by p38a from mice bearing 344SQR compared to 344SQP. FIG. 2J provides analysis of the level of cytokines and chemokines on TILs isolated from BMP7 knockdown tumors compared to control. FIG. 2K provides immunofluorescence analysis of p38a and SMAD1/5/9 phosphorylation in the macrophage cell line RAW 264.7 at 24 hours after treatment with BMP7 or BMP7 plus follistatin (foll). Scale bar, 100 μm (40× magnification).

FIGS. 3A-3K. BMP7 reduced macrophage-mediated pro-inflammatory signaling via p38α. In FIG. 3A, BMP7-knockdown and -control cells (0.5×10⁶) were injected into 129Sv/Ev mice and treated with anti-PD1 twice a week for 2 weeks. A week after the final anti-PD1 treatment, TILs were collected (n=3 for each group), and expression of p38α, IL-1α, IL-1β, TNF-α, RANTES, IFN-γ, and IL-2 were analyzed by qPCR. CD45 expression was used as a housekeeping gene for qPCR analysis. FIG. 3B provides qPCR analysis of p38α, IL-1α, IL-1β, TNF-α, and RANTES expression in RAW 264.7 cells transfected with siRNAs targeting p38a for 24 hours. FIG. 3C provides BMP7 levels in cell culture supernatant from 344SQP, 344SQR, and 344SQR ctrl and 344SQR shBMP7 cells analyzed by enzyme-linked immunosorbent assay. FIG. 3D provides quantitative PCR analysis of p38α, IL-1α, IL-1β, TNF-α, and RANTES expression in RAW 264.7 cells co-cultured with 344SQP or 344SQR, and 344SQR shBMP7 or 344SQR ctrl cells for 24 hours. FIG. 3E provides quantitative PCR analysis of p38α, IL-1α, IL-1β, TNF-α, and RANTES expression in RAW 264.7 cells co-cultured with 344SQR with or without the BMP7 receptor inhibitor K02288. FIG. 2F provides quantitative PCR analysis of p38α, IL-1α, IL-1β, TNF-α, and RANTES expression in peritoneal macrophages (PMD) co-cultured with 344SQR with or without the BMP7 receptor inhibitor K02288. FIGS. 3G and 3H provide qPCR analysis of p38α, IL-1α, IL-1β, TNF-α, and RANTES expression in RAW 264.7 cells and peritoneal macrophages (PMD) treated with BMP7 (FIG. 3G) or BMP7 plus follistatin (foll) (FIG. 3H) for 24 or 48 hours. FIGS. 3I and 3J provide qPCR analysis of p38α, IL-1α, IL-1β, TNF-α, and RANTES expression in RAW 264.7 cells (FIG. 3I) and peritoneal macrophages (PMD) (FIG. 3J) co-cultured with 344SQR cells or 344SQR cells plus follistatin (foll) for 24 or 48 hours. For FIGS. 3A-3J, P values are from unpaired, two-sided t tests, and error bars represent s.d. from two independent experiments. Statistical significance was defined as P<0.05. FIG. 3K provides expression analysis of TNF-α, IL1-β, and CD206 in RAW 264.7 cells that were either untreated, treated with BMP7, treated with siRNAs targeting p38α, or treated with siRNAs targeting p38a and BMP7.

FIGS. 4A-4G. BMP7 regulates CD4⁺ T cell production of IFN-γ and IL-2 via p38α. FIG. 4A provides Western blotting analysis of p38a and SMAD1/5/9 phosphorylation in CD4⁺ T cells at 1 hour after treatment with BMP7 or BMP7 plus follistatin (foll). ACTB expression was used for normalization in western blotting. In FIG. 4B, BMP7-knockdown and -control cells (0.5×10⁶) were injected into 129Sv/Ev mice and treated with anti-PD1 twice a week for 2 weeks. A week after the final anti-PD1 treatment, TILs were collected (n=3 for each group), and expression of IFN-γ, and IL-2 were analyzed by qPCR. CD45 expression was used as a housekeeping gene for qPCR analysis. FIG. 4C provides qPCR analysis of p38α, IFN-γ and IL-2 expression in EL4 cells stably overexpressing shRNAs targeting p38a (EL4 shp38α) compared with control (EL4 ctrl) cells. FIGS. 4D and 4E provide qPCR analysis of p38α, IFN-γ, and IL-2 expression in CD4+ T cells co-cultured with 344SQP or 344SQR (FIG. 4D), and 344SQR shBMP7 or 344SQR ctrl (FIG. 4E) cells for 24 hours. FIG. 4F provides qPCR analysis of p38α, IFN-γ, and IL-2 expression in CD4⁺ T cells treated with BMP7 or BMP7 plus follistatin (foll) for 24 hours. FIG. 4G provides qPCR analysis of p38α, IFN-γ, and IL-2 expression in CD4⁺ T cells co-cultured with 344SQR cells or 344SQR cells plus follistatin (foll) for 24 hours. For FIGS. 4B-4G, P values are from unpaired, two-sided t tests, and error bars represent s.d. from two independent experiments.

FIGS. 5A-5J. Inhibition of BMP7 expression re-sensitizes resistant tumors to anti-PD1 therapy. FIG. 5A provides tumor growth and survival analysis of mice with 344SQR ctrl (n=5) or 344SQR-shBMP7 (n=5) tumors treated with IgG or anti-PD1 (10 mg/kg) twice a week for 2 weeks. For the tumor growth graph, at the 7 and 9 day marks, the lines represent, from top to bottom: ctrl+IgG, ctrl+antiPD1, shBMP7+IgG, and shBMP7+antiPD1. For the survival graph, at the 20 day mark, the lines represent, from top to bottom, shBMP7+antiPD1, shBMPy+IgG, and ctrl+antiPD1. FIG. 5B provides tumor growth and survival analysis of mice with 4T1 or 4T1-shBMP7 tumors treated with IgG or anti-PD1. For the tumor growth graph, at the 15 day mark, the lines represent, from top to bottom: ctrl+antiPD1, shBMP7+IgG, ctrl+IgG, and shBMP7+antiPD1. For the survival graph, the lines that intersect the x-axis at the 20 day mark are ctrl+antiPD1 and shBMP7+IgG; the line that does not intersect the x-axis is shBMP7+anti-PD1. FIG. 5C provides tumor growth and survival analysis of mice with 344SQR tumors (n=5) treated with IgG, anti-PD1 (10 mg/kg), follistatin (0.1 mg/kg), or follistatin (0.1 mg/kg) plus anti-PD1 (10 mg/kg) for 2 weeks. For the tumor growth graph, at the 7 day mark, the lines represent, from top to bottom: IgG, anti-PD1, foll, and foll+anti-PD1. For the survival graph, the line that intersects the x-axis at the 20 day mark is IgG; the line that does not intersect the x-axis is foll+anti-PD1. For FIGS. 5A-5C, a two-way analysis of variance was used to compare tumor growth curves between groups. Mouse survival rates were analyzed by the Kaplan-Meier method and compared with log-rank tests. FIGS. 5D-5F provides flow cytometry analysis of CD8⁺ (FIG. 5D), CD8⁺IFN-γ⁺ (FIG. 5D), F4/80⁺CD206⁺ (FIG. 5E), CD4⁺ (FIG. 5F), and CD4⁺IFN-γ⁺ (FIG. 5F) T cells in tumor-infiltrating lymphocytes (TILs) from 344SQR ctrl (n=3) and 344SQR-shBMP7 (n=3) tumors treated with IgG or anti-PD1 (10 mg/kg) twice a week for 2 weeks. P values are from unpaired, two-sided t tests, and error bars represent s.d. for two independent experiments. FIGS. 5G and 5H provide representative images of immunohistochemical stains for CD206 (M2 macrophage marker) (FIG. 5G) and CD4 (FIG. 5H) in formalin-fixed paraffin-embedded tissue sections from BMP7-knockdown tumors treated with IgG or anti-PD1 compared with control. Scale bar, 100 μm (40× magnification). FIGS. 5I and 5J provide survival analyses of mice with 344SQR ctrl tumors (n=5) or 344SQR shBMP7 tumors (n=5) treated with IgG or anti-PDL1 (10 mg/kg) (FIG. 5I) or anti-CTLA4 (10 mg/kg) (FIG. 5J) twice a week for 2 weeks. For FIG. 5I, the lines intersecting the x-axis are, from left to right, ctrl+IgG, ctrl+antiPDL1, shBMP7+IgG, and shBMP7+antiPDL1. For FIG. 5J, the lines intersecting the x-axis are, from left to right, ctrl+IgG, ctrl+antiCTLA-4, shBMP7+IgG, and shBMP7+antiCTLA-4. Mouse survival rates were analyzed with the Kaplan-Meier method, and curves compared with log-rank tests.

### DETAILED DESCRIPTION

Although anti-PD1 drugs have produced durable control in some patients, about 80% of non-small cell lung cancer patients (NSCLC) do not respond to this therapy and even those who do often develop resistance. The mechanisms underlying immunosuppression and resistance to PD1 inhibitors in lung cancer are not well understood. Overexpression of BMP7, a member of TGF-beta superfamily, provides a mechanism for acquired resistance to anti-PD1 therapy in preclinical models and in patients with disease progression on immunotherapies. BMP7 is secreted by tumor cells and acts on macrophages and CD4⁺ T cells in the tumor microenvironment, promoting SMAD1/p38alpha signaling downregulation and impairment of pro-inflammatory responses. Knockdown of BMP7 or its neutralization via follistatin in combination with anti-PD1 re-sensitizes resistant tumors immunotherapies. Thus, BMP7 is an immunotherapeutic target in lung cancer.

### I. ASPECTS OF THE PRESENT INVENTION

The mechanisms of resistance to immunotherapies remains largely unknown. In this study, BMP7 was identified as a new regulator of resistance to immunotherapies. BMPs are secreted proteins that belong to the TGF-β superfamily and regulate proliferation, differentiation, and apoptosis in many different cell types, including immune cells. Binding of BMP to its receptor leads to the phosphorylation of intracellular Smads, which then bind to co-Smad 4 and translocate into the nucleus to regulate gene expression. Treatment with members of the BMP family in vitro and in vivo significantly enhanced monocyte polarization into M2-macrophages (Rocher et al., 2012; Singla et al., 2016; Rocher & Singla, 2013). BMP has been shown to regulate activation, growth, and cytokine secretion in macrophages (Hong et al., 2009; Kwon et al., 2009), including IL-10 (Lee et al., 2013; Owens et al., 2015). In addition, previous studies have shown that BMPs promote PD-L1 and PD-L2 upregulation in dendritic cells (DCs) (Martinez et al., 2011). BMP7 and other BMPs have been shown to also signal via p38a in a dose-dependent manner (Hu et al., 2004; Lee et al., 2002; Iwasaki et al., 1999; Awazu et al., 2017; Wang et al., 2016; Takahashi et al., 2008). P38 appears to play a critical role in regulation of the expression of a number of proinflammatory chemokines and cytokines induced by IFN-λ, (Valledor et al., 2008). P38 proteins are a class of mitogen-activated protein kinases (MAPKs) that are major players during inflammatory responses, especially in macrophages (Yang et al., 2012). P38a is the critical isoform in inflammatory responses and is involved in the expression of proinflammatory mediators in macrophages such as IL-1β, TNF-α and IL-12 (Yang et al., 2012; Byeon et al., 2011; Garcia et al., 1998) as well as COX-2, IL-8, IL-6, IL-3, IL-2, and IL-1, all of which contain AU-rich elements (AREs) in their 3′ untranslated regions to which p38 binds (Amirouche et al., 2013).

BMP7 was upregulated in mouse and human tumors resistant to anti-PD1 therapy, and BMP7 levels were higher not only in blood from mice bearing resistant tumors but also in pretreatment blood from patients with disease progression on anti-PD1 and radiotherapy. Also, BMP7 levels were analyzed in plasma from PD patients before pembrolizumab and radiotherapy and at progression, but BMP7 levels were no different in those samples. These studies reveal that secreted BMP7 impinges effector T cell functions while favoring the generation of immunosuppressive cells precluding response to immunotherapy. Collectively, targeting BMP7 represents a new approach to overcome resistance to checkpoint blockade therapies in cancer.

Previously, a preclinical NSCLC model (p53^{R172HΔg}/+K-ras^{LA1/+}) with acquired resistance to anti-PD1 was generated in a syngeneic host repeatedly dosed with anti-mouse PD1 antibodies (Wang et al., 2016). After profiling tumors resistant to anti-PD1 compared to parental tumors, BMP7 was found to be the most upregulated gene in anti-PD1 resistant tumors (344SQR) compared to parental tumors (344SQP). These findings were validated using qPCR and IHC staining. Since BMPs are secreted, whether tumors resistant to anti-PD1 secreted BMP7 into the bloodstream was investigated. BMP7 levels were found to be higher in mice bearing resistant tumors compared to parental.

The BMP7 promoter was hypomethylated in anti-PD1-resistant tumors from the preclinical model, which explains its upregulated RNA and protein levels. Previous studies have also shown that BMP7 expression can be regulated via epigenetic mechanisms (Loeser et al., 2009; Kron et al., 2009). Interestingly, others have shown that epigenetic drugs targeting histone deacetylation or methylation modulate the immune response and overcome acquired resistance to immunotherapy. For example, epigenetic drugs enhance the efficacy of immune checkpoint inhibitor therapy by increasing the expression of immune checkpoint ligands and tumor-associated antigens on tumor cells (Dunn & Rao, 2017). The present results suggest that epigenetics drugs can also modulate genes that promote immunosuppression such as BMP7.

The present studies revealed that proteins known to be related to BMP7 signaling, such as p38a (Takahashi et al., 2008; Hu et al., 2004; Lee et al., 2002; Iwasaki et al., 1999; Awazu et al., 2017; Wang et al., 2016), were expressed differently in resistant tumors than in parental tumors. Because p38a was known to be regulated by BMP7 (Li et al., 2015; Takahashi et al., 2008) via SMAD activation (Hu et al., 2004), this protein was focused on for validation studies. BMP7 can either promote or inhibit p38 MAPK activation depending on the cellular context and BMP7 dose (Li et al., 2015; Takahashi et al., 2008; Hu et al., 2004; Awazu et al., 2017). Here, BMP7 specifically regulated p38a at the mRNA and protein levels. p38a downregulation was validated in 344SQP versus 344SQR tumors, and in cancer patients with progression on immunotherapies. SMAD1 activation status was also validated in patient samples with high BMP7 expression in IHC analysis. These findings suggest that BMP7 downregulates p38a via SMAD1 activation in tumors resistant to anti-PD1 therapy. p38a can act as a tumor suppressor by regulating cell cycle progression and induction of apoptosis or as an oncogene by promoting invasion, inflammation, and angiogenesis (Wagner & Nebreda, 2009).

p38a was downregulated not only in tumors but also in TILs from anti-PD1-resistant tumors versus parental tumors. p38 proteins are important participants in inflammatory responses and are activated in response to a variety of cellular stresses including osmotic shock, lipopolysaccharides, and inflammatory cytokines (Lee et al., 1994; Kim et al., 2004; Zhu et al., 2000; Baldassare et al., 1999; Yang et al., 2014). p38a is the critical isoform in inflammatory responses and is involved in the expression of pro-inflammatory mediators in macrophages such as IL-1β, TNF-α, and IL-12 (Yang et al., 2012; Byeon et al., 2011; Garcia et al., 1998) as well as RANTES (Valledor et al., 2008), COX-2, IL-8, IL-6, IL-3, IL-2, and IL-1, all of which contain AU-rich elements in their 3′ untranslated regions to which p38 binds (Amirouche et al., 2013). p38a participates in the regulation of IFN-γ expression and its mRNA stabilization in immune cells (Rincon et al., 1998; Mavropoulos et al., 2005). Strikingly, the p38α-regulated inflammatory cytokines IL-1α, IL-1β, and TNF-α were downregulated in TILs collected from 344SQR tumors treated with anti-PD1 versus 344SQP. Cytokines and chemokines regulated by p38a including IL-1α, IL-1β, TNF-α, RANTES, IFN-γ, and IL-2 were also downregulated in blood from mice bearing 344SQR tumors compared with parental tumors. Others have also found that BMP7 treatment led to significant reductions in pro-inflammatory cytokines, including TNF-α, in macrophages in in vivo (Rocher et al., 2012; Shoulders et al., 2018), and that BMP7 represses TNF-α and IL-1β in models of chronic and acute renal failure and in chondrocytes from patients with osteoarthritis (Gould et al., 2002; Gavenis et al., 2011). The present findings confirmed that p38α, IL-1α, IL-1β, TNF-α, RANTES, IFN-γ, and IL-2 expression levels were increased in TILs isolated from BMP7-knockdown tumors compared with control. These results suggest that BMP7 regulates p38a expression not only in tumors resistant to anti-PD1 but also in TILs in the tumor microenvironment, and that BMP7 also regulates expression of proinflammatory cytokines and chemokines in TILs via p38a regulation. Next, to investigate whether BMP7 regulates p38a via SMAD1 activation in immune cells, as was seen in tumors, macrophages and CD4⁺ T cells were treated with BMP7, with or without its natural inhibitor follistatin, and SMAD1/5/9 activation analyzed. The results suggest that BMP7 also regulates p38a through SMAD1 activation in these cells. These findings are supported by previous studies in macrophages isolated from a vivo model of atherosclerosis treated with intravenous injections of BMP7 or liposomal clodronate (Shoulders et al., 2018). In that study, BMP7 significantly reduced the number of proinflammatory macrophages and decreased p38 activation while increasing SMAD1/5/8 phosphorylation in macrophages. Other studies also showed that BMP7 promotes M2 polarization in human and mouse macrophages in vitro and in vivo models (Rocher et al., 2012; Singla et al., 2016; Rocher et al., 2013).

p38 MAPK signaling promotes not only M2 monocytes polarization into M1-type cells in cells treated with lipopolysaccharides (Yang et al., 2013) but also is central in the activation of pro-inflammatory gene transcription. In macrophages, p38a is activated by lipopolysaccharide and Toll-like receptor-4, which subsequently activates pro-inflammatory cytokines, including interleukin (IL)-1 and tumor necrosis factor (TNF)-α (Lee et al., 1994; Kim et al., 2004; Zhu et al., 2000; Baldassare et al., 1999). Therefore, whether secreted BMP7 reduced pro-inflammatory and chemokines in via p38a in macrophages was investigated. Murine macrophages co-cultured with 344SQR cells had lower expression of p38α, IL-1α, IL-1β, TNF-α, and RANTES compared with cells co-cultured with 344SQP cells. On the other hand, murine macrophages co-cultured with BMP7-knockdown 344SQR cells expressed higher levels of p38α, IL-1α, IL-1β, TNF-α, and RANTES compared with 344SQR ctrl cells. That these findings depended on BMP7 was confirmed by treating murine macrophages and peritoneal macrophages with BMP7, with or without follistatin. As expected, both macrophage types expressed higher levels of p38α, IL-1α, TNF-α, IL-1β, and RANTES when treated with BMP7 plus follistatin compared with BMP7 only. Treatment of 344SQR cells with follistatin led to similar results. Taken together these findings suggest that BMP7 suppresses the pro-inflammatory cytokine expression regulated by p38a in macrophages.

p38 signaling is known to be activated in T cells stimulated via TCR signaling and reduced in anergic T cells (DeSilva et al., 1997). p38a also participates in the regulation of IFN-γ expression in CD4+ T cells (Rincon et al., 1998) and promotes the 3′-untranslated region stabilization of IFN-γ mRNA in NK cells (Mavropoulos et al., 2005). Further, the inhibition of p38 MAPK in Th1 cells differentiated in vitro blocked the IFN-γ expression induced by IL-12/IL-18 and CD3/CD28 stimulation (Yang et al., 2001; Yu et al., 2003; Zhang et al., 1999). Notably, in the present study, IL-12p70 and IL-12p40 levels were lower in blood from mice bearing 344SQR tumors versus parental tumors. Previous studies showed that treating cells with SB203580, a specific inhibitor of p38, suppressed the transcriptional activation of the IL-2 promoter in T lymphocytes (Matsuda et al., 1998). Therefore, the effect of BMP7 on IFN-γ and IL-2 in T cells was investigated. SMAD regulatory pathways regulate different aspects of immune activation and immune suppression in T cells (Malhotra & Kang, 2013). For example, TGF-β promotes the differentiation of CD4⁺ T cells in the suppressive FOXP3⁺ T regulatory cells via SMAD activation (Takimoto et al., 2010). In the present study, it was found that BMP7 regulates p38a expression via SMAD1 signaling not only in tumors and macrophages but also in CD4⁺ T cells. In addition, it was found that BMP7 regulates the expression of IFN-γ and IL-2 in a p38α-dependent manner. Activated CD4⁺ T cells with BMP7 plus follistatin had higher p38α, IFN-γ and IL-2 expression versus BMP7 only. In agreement with these findings, inhibiting p38a activity with the specific inhibitor SB203580 led to suppressed T-cell proliferation in response to IL-2 (Crawley et al., 1997). Other studies have also correlated IL-2 activation with p38 MAPK signaling in T cells (Kogkopoulou et al., 2006; Veiopoulou et al., 2004; Nguyen et al., 2000). Notably, other BMPs can promote or inhibit T-cell proliferation and IFN-γ and IL-2 production (Chen & Ten Dijke, 20160. Indeed, BMP2, BMP4, and BMP6 can promote CD4+ T-cell proliferation and IL-2 production (Martinez et al., 2015). In this study, it was found that BMP7 decreased IFN-γ and IL-2 expression in CD4⁺ T cells via p38α.

Finally, whether BMP7 knockdown or its neutralization via follistatin could re-sensitize anti-PD1-resistant tumors to immunotherapy was tested. BMP7 knockdown and treatment with follistatin re-sensitized tumors to anti-PD1 and extended survival relative to the control. Since follistatin not only neutralizes BMP7 but other members of the TGF-β superfamily such as activins, it might represent a broader approach to overcome resistance to anti-PD1. Interestingly, the combination of BMP7 knockdown and anti-CTLA4 or anti-PDL1 also extended survival compared with control, leading to the evaluation of whether mechanisms of resistance to anti-PD1 overlap with resistance to anti-CTLA4 or anti-PDL1. Increased numbers of CD4⁺ T cells in BMP7-knockdown tumors treated with anti-PD1 or IgG compared with control were also found. CD4⁺IFN-γ⁺ T cells were higher in BMP7-knockdown tumors treated with anti-PD1 or IgG than in control tumors treated with IgG. Increased numbers and activation of CD8⁺ T cells in BMP7-knockdown tumors treated with anti-PD1 was found. On the other hand, BMP7-knockdown tumors treated with IgG or anti-PD1 had decreased percentages of M2 macrophages compared with control tumors treated with IgG or anti-PD1. These findings are supported by others showing that BMP7 increases M2 macrophage differentiation in vitro and in vivo in different models (Rocher et al., 2012; Singla et al., 2016; Rocher et al., 2013; Shoulders et al., 2018).

Secreted BMP7 promotes resistance to anti-PD1 therapy by repressing macrophage-mediated inflammatory responses and Th1-associated cytokines in the tumor microenvironment. BMP7 downregulated p38a and p38-regulated cytokines and chemokines including IL-1α, IL-1β, TNF-α, and RANTES via SMAD1 activation. At the same time, BMP7 decreased CD4⁺ T-cell activation by downregulating IFN-γ and IL-2 expression via SMAD1/p38a signaling (FIG. 6F). BMP7 inhibition represents a new target for overcoming resistance to cancer immunotherapies.

### II. INHIBITION OF BMP7

A. BMP7 Small Molecule Inhibitors

One strategy for inhibiting the function of BMP7 involves the use of small molecule inhibitors to prevent the binding of BMP7 to its receptors. Such BMP7 inhibitors may function by binding to the BMP7 receptor and inhibiting its function. Exemplary BMP inhibitors include 3-[ (6-Amino-5-(3,4,5-trimethoxyphenyl)-3-pyridinyl]phenol (K02288), Quinoline, 5-[6-(4-methoxyphenyl)pyrazolo[1,5-a]pyrimidin-3-yl](ML347, LDN-193719), 1-(4-(6-methyl-5-(3,4,5-trimethoxyphenyl)pyridin-3-yl)phenyl)piperazine (LDN-214117), Quinoline, 5-[6-[4-(1-piperazinyl)phenyl]pyrazolo[1,5-a]pyrimidin-3-yl](LDN-212854), 4-(6-(4-(piperazin-1-yl)phenyl)pyrazolo[1,5-a]pyrimidin-3-yl)quinoline hydrochloride (LDN193189), 6-[4-[2-(1-Piperidinyl)ethoxy]phenyl]-3-(4-pyridinyl)-pyrazolo[1,5-a]pyrimidine dihydrochloride (Dorsomorphin), 4-[6-[4-(1-Methylethoxy)phenyl]pyrazolo[1,5-a]pyrimidin-3-yl]-quinoline (DMH1), 4-[6-[4-[2-(4-Morpholinyl)ethoxy]phenyl]pyrazolo[1,5-a]pyrimidin-3-yl]quinoline (DMH-2), and 5-[6-(4-Methoxyphenyl)pyrazolo[1,5-a]pyrimidin-3-yl]quinoline (ML 347).

B. BMP7 Antagonistic Proteins

Another strategy for inhibiting the function of BMP7 involves the use of proteinaceous molecules known to inhibit the function of BMP-7. Exemplary BMP7 antagonists include follistatin, noggin (NOG), and uterine sensitization-associated gene-1 (USAG-1). The BMP7 antagonistic protein may be PEGylated. PEGylation is the process of covalent attachment of poly(ethylene glycol) polymer chains to another molecule, normally a drug or therapeutic protein. PEGylation is routinely achieved by incubation of a reactive derivative of PEG with the target macromolecule. The covalent attachment of PEG to a drug or therapeutic protein can “mask” the agent from the host's immune system (reduced immunogenicity and antigenicity) or increase the hydrodynamic size (size in solution) of the agent, which prolongs its circulatory time by reducing renal clearance. PEGylation can also provide water solubility to hydrophobic drugs and proteins.

The first step of the PEGylation is the suitable functionalization of the PEG polymer at one or both terminals. PEGs that are activated at each terminus with the same reactive moiety are known as “homobifunctional,” whereas if the functional groups present are different, then the PEG derivative is referred as “heterobifunctional” or “heterofunctional.” The chemically active or activated derivatives of the PEG polymer are prepared to attach the PEG to the desired molecule.

The choice of the suitable functional group for the PEG derivative is based on the type of available reactive group on the molecule that will be coupled to the PEG. For proteins, typical reactive amino acids include lysine, cysteine, histidine, arginine, aspartic acid, glutamic acid, serine, threonine, and tyrosine. The N-terminal amino group and the C-terminal carboxylic acid can also be used.

The techniques used to form first generation PEG derivatives are generally reacting the PEG polymer with a group that is reactive with hydroxyl groups, typically anhydrides, acid chlorides, chloroformates, and carbonates. In the second generation PEGylation chemistry more efficient functional groups, such as aldehyde, esters, amides, etc., are made available for conjugation.

As applications of PEGylation have become more and more advanced and sophisticated, there has been an increase in need for heterobifunctional PEGs for conjugation. These heterobifunctional PEGs are very useful in linking two entities, where a hydrophilic, flexible, and biocompatible spacer is needed. Preferred end groups for heterobifunctional PEGs are maleimide, vinyl sulfones, pyridyl disulfide, amine, carboxylic acids, and NHS esters.

The most common modification agents, or linkers, are based on methoxy PEG (mPEG) molecules. Their activity depends on adding a protein-modifying group to the alcohol end. In some instances, polyethylene glycol (PEG diol) is used as the precursor molecule. The diol is subsequently modified at both ends in order to make a hetero- or homo-dimeric PEG-linked molecule.

Proteins are generally PEGylated at nucleophilic sites, such as unprotonated thiols (cysteinyl residues) or amino groups. Examples of cysteinyl-specific modification reagents include PEG maleimide, PEG iodoacetate, PEG thiols, and PEG vinylsulfone. All four are strongly cysteinyl-specific under mild conditions and neutral to slightly alkaline pH but each has some drawbacks. The thioether formed with the maleimides can be somewhat unstable under alkaline conditions so there may be some limitation to formulation options with this linker. The carbamothioate linkage formed with iodo PEGs is more stable, but free iodine can modify tyrosine residues under some conditions. PEG thiols form disulfide bonds with protein thiols, but this linkage can also be unstable under alkaline conditions. PEG-vinylsulfone reactivity is relatively slow compared to maleimide and iodo PEG; however, the thioether linkage formed is quite stable. Its slower reaction rate also can make the PEG-vinylsulfone reaction easier to control.

Site-specific PEGylation at native cysteinyl residues is seldom carried out, since these residues are usually in the form of disulfide bonds or are required for biological activity. On the other hand, site-directed mutagenesis can be used to incorporate cysteinyl PEGylation sites for thiol-specific linkers. The cysteine mutation must be designed such that it is accessible to the PEGylation reagent and is still biologically active after PEGylation.

Amine-specific modification agents include PEG NHS ester, PEG tresylate, PEG aldehyde, PEG isothiocyanate, and several others. All react under mild conditions and are very specific for amino groups. The PEG NHS ester is probably one of the more reactive agents; however, its high reactivity can make the PEGylation reaction difficult to control on a large scale. PEG aldehyde forms an imine with the amino group, which is then reduced to a secondary amine with sodium cyanoborohydride. Unlike sodium borohydride, sodium cyanoborohydride will not reduce disulfide bonds. However, this chemical is highly toxic and must be handled cautiously, particularly at lower pH where it becomes volatile.

Due to the multiple lysine residues on most proteins, site-specific PEGylation can be a challenge. Fortunately, because these reagents react with unprotonated amino groups, it is possible to direct the PEGylation to lower-pK amino groups by performing the reaction at a lower pH. Generally, the pK of the alpha-amino group is 1-2 pH units lower than the epsilon-amino group of lysine residues. By PEGylating the molecule at pH 7 or below, high selectivity for the N-terminus frequently can be attained. However, this is only feasible if the N-terminal portion of the protein is not required for biological activity. Still, the pharmacokinetic benefits from PEGylation frequently outweigh a significant loss of in vitro bioactivity, resulting in a product with much greater in vivo bioactivity regardless of PEGylation chemistry.

There are several parameters to consider when developing a PEGylation procedure. Fortunately, there are usually no more than four or five parameters. The “design of experiments” approach to optimization of PEGylation conditions can be very useful. For thiol-specific PEGylation reactions, parameters to consider include: protein concentration, PEG-to-protein ratio (on a molar basis), temperature, pH, reaction time, and in some instances, the exclusion of oxygen. (Oxygen can contribute to intermolecular disulfide formation by the protein, which will reduce the yield of the PEGylated product.) The same factors should be considered (with the exception of oxygen) for amine-specific modification except that pH may be even more critical, particularly when targeting the N-terminal amino group.

For both amine- and thiol-specific modifications, the reaction conditions may affect the stability of the protein. This may limit the temperature, protein concentration, and pH. In addition, the reactivity of the PEG linker should be known before starting the PEGylation reaction. For example, if the PEGylation agent is only 70% active, the amount of PEG used should ensure that only active PEG molecules are counted in the protein-to-PEG reaction stoichiometry

C. BMP7 Neutralizing Antibodies

A third strategy involves the use of antibodies that neutralize BMP7. Examples of antibodies that neutralize BMP7 include ABIN1100288 from Antibodies-Online, LS-C149884-25 from Lifespan Biosciences, GTX52570 from Genetex, and GTX31164 from Genetex. Antibodies according to the present disclosure may be defined, in the first instance, by their binding specificity. Those of skill in the art, by assessing the binding specificity/affinity of a given antibody using techniques well known to those of skill in the art, can determine whether such antibodies fall within the scope of the instant claims. For example, the epitope to which a given antibody bind may consist of a single contiguous sequence of 3 or more (e.g., 3, 4, 5, 6, 7, 8, 9, 10, 11, 12, 13, 14, 15, 16, 17, 18, 19, 20) amino acids located within the antigen molecule (e.g., a linear epitope in a domain). Alternatively, the epitope may consist of a plurality of non-contiguous amino acids (or amino acid sequences) located within the antigen molecule (e.g., a conformational epitope).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, N.Y.). Cross-blocking can be measured in various binding assays such as ELISA, biolayer interferometry, or surface plasmon resonance. Other methods include alanine scanning mutational analysis, peptide blot analysis (Reineke (2004) Methods Mol. Biol. 248: 443-63), peptide cleavage analysis, high-resolution electron microscopy techniques using single particle reconstruction, cryoEM, or tomography, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267: 252-259; Engen and Smith (2001) Anal. Chem. 73: 256A-265A.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation. Here, the preferred epitope is a conformational epitope that is present in homotrimeric type I collagen but absent in heterotrimeric type I collagen.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (see US 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. MAP may be used to sort the antibodies of the disclosure into groups of antibodies binding different epitopes.

The present disclosure includes antibodies that may bind to the same epitope, or a portion of the epitope. Likewise, the present disclosure also includes antibodies that compete for binding to a target or a fragment thereof with any of the specific exemplary antibodies described herein. One can easily determine whether an antibody binds to the same epitope as, or competes for binding with, a reference antibody by using routine methods known in the art. For example, to determine if a test antibody binds to the same epitope as a reference, the reference antibody is allowed to bind to target under saturating conditions. Next, the ability of a test antibody to bind to the target molecule is assessed. If the test antibody is able to bind to the target molecule following saturation binding with the reference antibody, it can be concluded that the test antibody binds to a different epitope than the reference antibody. On the other hand, if the test antibody is not able to bind to the target molecule following saturation binding with the reference antibody, then the test antibody may bind to the same epitope as the epitope bound by the reference antibody.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art. Structural studies with EM or crystallography also can demonstrate whether or not two antibodies that compete for binding recognize the same epitope.

In another aspect, the antibodies may be defined by their variable sequence, which include additional “framework” regions. Furthermore, the antibodies sequences may vary from these sequences, optionally using methods discussed in greater detail below. For example, nucleic acid sequences may vary from those set out above in that (a) the variable regions may be segregated away from the constant domains of the light and heavy chains, (b) the nucleic acids may vary from those set out above while not affecting the residues encoded thereby, (c) the nucleic acids may vary from those set out above by a given percentage, e.g., 70%, 75%, 80%, 85%, 90%, 91%, 92%, 93%, 94%, 95%, 96%, 97%, 98% or 99% homology, (d) the nucleic acids may vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C., (e) the amino acids may vary from those set out above by a given percentage, e.g., 80%, 85%, 90%, 91%, 92%, 93%, 94%, 95%, 96%, 97%, 98% or 99% homology, or (f) the amino acids may vary from those set out above by permitting conservative substitutions (discussed below).

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogeny pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80: 726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One particular example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the disclosure. Software for performing BLAST analyses is publicly available through the National Center for Biotechnology Information. The rearranged nature of an antibody sequence and the variable length of each gene requires multiple rounds of BLAST searches for a single antibody sequence. Also, manual assembly of different genes is difficult and error-prone. The sequence analysis tool IgBLAST (world-wide-web at ncbi.nlm.nih.gov/igblast/) identifies matches to the germline V, D and J genes, details at rearrangement junctions, the delineation of Ig V domain framework regions and complementarity determining regions. IgBLAST can analyze nucleotide or protein sequences and can process sequences in batches and allows searches against the germline gene databases and other sequence databases simultaneously to minimize the chance of missing possibly the best matching germline V gene.

In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

Yet another way of defining an antibody is as a “derivative” of any of the below-described antibodies and their antigen-binding fragments. The term “derivative” refers to an antibody or antigen-binding fragment thereof that immunospecifically binds to an antigen but which comprises, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. The term “derivative” encompasses, for example, as variants having altered CH1, hinge, CH2, CH3 or CH4 regions, so as to form, for example antibodies, etc., having variant Fc regions that exhibit enhanced or impaired effector or binding characteristics. The term “derivative” additionally encompasses non-amino acid modifications, for example, amino acids that may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment, the altered carbohydrate modifications enhance antibody mediated effector function relative to the antibody lacking the carbohydrate modification. Carbohydrate modifications that lead to altered antibody mediated effector function are well known in the art (for example, see Shields, R. L. et al. (2002) “Lack Of Fucose On Human IgG N-Linked Oligosaccharide Improves Binding To Human Fcgamma RIII And Antibody-Dependent Cellular Toxicity,” J. Biol. Chem. 277(30): 26733-26740; Davies J. et al. (2001) “Expression Of GnTIII In A Recombinant Anti-CD20 CHO Production Cell Line: Expression Of Antibodies With Altered Glycoforms Leads To An Increase In ADCC Through Higher Affinity For FC Gamma RIII,” Biotechnology & Bioengineering 74(4): 288-294). Methods of altering carbohydrate contents are known to those skilled in the art, see, e.g., Wallick, S. C. et al. (1988) “Glycosylation Of A VH Residue Of A Monoclonal Antibody Against Alpha (1----6) Dextran Increases Its Affinity For Antigen,” J. Exp. Med. 168(3): 1099-1109; Tao, M. H. et al. (1989) “Studies Of Aglycosylated Chimeric Mouse-Human IgG. Role Of Carbohydrate In The Structure And Effector Functions Mediated By The Human IgG Constant Region,” J. Immunol. 143(8): 2595-2601; Routledge, E. G. et al. (1995) “The Effect Of Aglycosylation On The Immunogenicity Of A Humanized Therapeutic CD3 Monoclonal Antibody,” Transplantation 60(8):847-53; Elliott, S. et al. (2003) “Enhancement Of Therapeutic Protein In Vivo Activities Through Glycoengineering,” Nature Biotechnol. 21:414-21; Shields, R. L. et al. (2002) “Lack Of Fucose On Human IgG N-Linked Oligosaccharide Improves Binding To Human Fcgamma RIII And Antibody-Dependent Cellular Toxicity,” J. Biol. Chem. 277(30): 26733-26740).

A derivative antibody or antibody fragment can be generated with an engineered sequence or glycosylation state to confer preferred levels of activity in antibody dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

D. BMP7 Antisense Oligonucleotides

Another strategy involves the use of antisense oligonucleotides to knockdown the expression of BMP7. The antisense oligonucleotides may be complementary to specific regions of the BMP7 mRNA (NCBI Reference Sequence NM_001719.2, which is incorporated by reference herein). siNA (e.g., siRNA) are well known in the art. For example, siRNA and double-stranded RNA have been described in U.S. Pat. Nos. 6,506,559 and 6,573,099, as well as in U.S. Patent Applications 2003/0051263, 2003/0055020, 2004/0265839, 2002/0168707, 2003/0159161, and 2004/0064842, all of which are herein incorporated by reference in their entirety.

Within a siNA, the components of a nucleic acid need not be of the same type or homogenous throughout (e.g., a siNA may comprise a nucleotide and a nucleic acid or nucleotide analog). Typically, siNA form a double-stranded structure; the double-stranded structure may result from two separate nucleic acids that are partially or completely complementary. In certain embodiments of the present invention, the siNA may comprise only a single nucleic acid (polynucleotide) or nucleic acid analog and form a double-stranded structure by complementing with itself (e.g., forming a hairpin loop). The double-stranded structure of the siNA may comprise 16, 20, 25, 30, 35, 40, 45, 50, 60, 65, 70, 75, 80, 85, 90, 100, 150, 200, 250, 300, 350, 400, 450, 500 or more contiguous nucleobases, including all ranges therein. The siNA may comprise 17 to 35 contiguous nucleobases, more preferably 18 to 30 contiguous nucleobases, more preferably 19 to 25 nucleobases, more preferably 20 to 23 contiguous nucleobases, or 20 to 22 contiguous nucleobases, or 21 contiguous nucleobases that hybridize with a complementary nucleic acid (which may be another part of the same nucleic acid or a separate complementary nucleic acid) to form a double-stranded structure.

Agents of the present invention useful for practicing the methods of the present invention include, but are not limited to siRNAs. Typically, introduction of double-stranded RNA (dsRNA), which may alternatively be referred to herein as small interfering RNA (siRNA), induces potent and specific gene silencing, a phenomena called RNA interference or RNAi. RNA interference has been referred to as “cosuppression,” “post-transcriptional gene silencing,” “sense suppression,” and “quelling.” RNAi is an attractive biotechnological tool because it provides a means for knocking out the activity of specific genes.

In designing RNAi there are several factors that need to be considered, such as the nature of the siRNA, the durability of the silencing effect, and the choice of delivery system. To produce an RNAi effect, the siRNA that is introduced into the organism will typically contain exonic sequences. Furthermore, the RNAi process is homology dependent, so the sequences must be carefully selected so as to maximize gene specificity, while minimizing the possibility of cross-interference between homologous, but not gene-specific sequences. Preferably the siRNA exhibits greater than 80%, 85%, 90%, 95%, 98%, or even 100% identity between the sequence of the siRNA and the gene to be inhibited. Sequences less than about 80% identical to the target gene are substantially less effective. Thus, the greater homology between the siRNA and the gene to be inhibited, the less likely expression of unrelated genes will be affected.

In addition, the size of the siRNA is an important consideration. In some embodiments, the present invention relates to siRNA molecules that include at least about 19-25 nucleotides and are able to modulate gene expression. In the context of the present invention, the siRNA is preferably less than 500, 200, 100, 50, or 25 nucleotides in length. More preferably, the siRNA is from about 19 nucleotides to about 25 nucleotides in length.

A target gene generally means a polynucleotide comprising a region that encodes a polypeptide, or a polynucleotide region that regulates replication, transcription, or translation or other processes important to expression of the polypeptide, or a polynucleotide comprising both a region that encodes a polypeptide and a region operably linked thereto that regulates expression. Any gene being expressed in a cell can be targeted. Preferably, a target gene is one involved in or associated with the progression of cellular activities important to disease or of particular interest as a research object.

siRNA can be obtained from commercial sources, natural sources, or can be synthesized using any of a number of techniques well-known to those of ordinary skill in the art. For example, one commercial source of predesigned siRNA is Ambion®, Austin, Tex. Another is Qiagen® (Valencia, Calif.). An inhibitory nucleic acid that can be applied in the compositions and methods of the present invention may be any nucleic acid sequence that has been found by any source to be a validated downregulator of a protein of interest. Without undue experimentation and using the disclosure of this invention, it is understood that additional siRNAs can be designed and used to practice the methods of the invention.

The siRNA may also comprise an alteration of one or more nucleotides. Such alterations can include the addition of non-nucleotide material, such as to the end(s) of the 19 to 25 nucleotide RNA or internally (at one or more nucleotides of the RNA). In certain aspects, the RNA molecule contains a 3′-hydroxyl group. Nucleotides in the RNA molecules of the present invention can also comprise non-standard nucleotides, including non-naturally occurring nucleotides or deoxyribonucleotides. The double-stranded oligonucleotide may contain a modified backbone, for example, phosphorothioate, phosphorodithioate, or other modified backbones known in the art, or may contain non-natural internucleoside linkages. Additional modifications of siRNAs (e.g., 2′-O-methyl ribonucleotides, 2′-deoxy-2′-fluoro ribonucleotides, “universal base” nucleotides, 5-C-methyl nucleotides, one or more phosphorothioate internucleotide linkages, and inverted deoxyabasic residue incorporation) can be found in U.S. Application Publication 2004/0019001 and U.S. Pat. No. 6,673,611 (each of which is incorporated by reference in its entirety). Collectively, all such altered nucleic acids or RNAs described above are referred to as modified siRNAs.

As exosomes are known to comprise DICER and active RNA processing RISC complex (see PCT Publn. WO 2014/152622, which is incorporated herein by reference in its entirety), shRNA transfected into exosomes can mature into RISC-complex bound siRNA with the exosomes themselves. Alternatively, mature siRNA can itself be transfected into exosomes or liposomes.

### III. LIPID-BASED NANOPARTICLES

In some embodiments, a lipid-based nanoparticle is a liposomes, an exosomes, lipid preparations, or another lipid-based nanoparticle, such as a lipid-based vesicle (e.g., a DOTAP:cholesterol vesicle). Lipid-based nanoparticles may be positively charged, negatively charged or neutral. Lipid-based nanoparticles may comprise the necessary components to allow for transcription and translation, signal transduction, chemotaxis, or other cellular functions. Lipid-based nanoparticles may be used to deliver small molecule drugs, protein-based therapeutics, nucleic-acid based therapeutics, or combinations thereof.

A. Liposomes

A “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition. Liposomes provided herein include unilamellar liposomes, multilamellar liposomes, and multivesicular liposomes. Liposomes provided herein may be positively charged, negatively charged, or neutrally charged. In certain embodiments, the liposomes are neutral in charge.

A multilamellar liposome has multiple lipid layers separated by aqueous medium. Such liposomes form spontaneously when lipids comprising phospholipids are suspended in an excess of aqueous solution. The lipid components undergo self-rearrangement before the formation of closed structures and entrap water and dissolved solutes between the lipid bilayers. Lipophilic molecules or molecules with lipophilic regions may also dissolve in or associate with the lipid bilayer.

In specific aspects, a polypeptide, a nucleic acid, or a small molecule drug may be, for example, encapsulated in the aqueous interior of a liposome, interspersed within the lipid bilayer of a liposome, attached to a liposome via a linking molecule that is associated with both the liposome and the polypeptide/nucleic acid, entrapped in a liposome, complexed with a liposome, or the like.

A liposome used according to the present embodiments can be made by different methods, as would be known to one of ordinary skill in the art. For example, a phospholipid, such as for example the neutral phospholipid dioleoylphosphatidylcholine (DOPC), is dissolved in tert-butanol. The lipid(s) is then mixed with a polypeptide, nucleic acid, and/or other component(s). Tween 20 is added to the lipid mixture such that Tween 20 is about 5% of the composition's weight. Excess tert-butanol is added to this mixture such that the volume of tert-butanol is at least 95%. The mixture is vortexed, frozen in a dry ice/acetone bath and lyophilized overnight. The lyophilized preparation is stored at −20° C. and can be used up to three months. When required the lyophilized liposomes are reconstituted in 0.9% saline.

Alternatively, a liposome can be prepared by mixing lipids in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min to 2 h, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

The dried lipids or lyophilized liposomes prepared as described above may be dehydrated and reconstituted in a solution of a protein or peptide and diluted to an appropriate concentration with a suitable solvent, e.g., DPBS. The mixture is then vigorously shaken in a vortex mixer. Unencapsulated additional materials, such as agents including but not limited to hormones, drugs, nucleic acid constructs and the like, are removed by centrifugation at 29,000×g and the liposomal pellets washed. The washed liposomes are resuspended at an appropriate total phospholipid concentration, e.g., about 50-200 mM. The amount of additional material or active agent encapsulated can be determined in accordance with standard methods. After determination of the amount of additional material or active agent encapsulated in the liposome preparation, the liposomes may be diluted to appropriate concentrations and stored at 4° C. until use. A pharmaceutical composition comprising the liposomes will usually include a sterile, pharmaceutically acceptable carrier or diluent, such as water or saline solution.

Additional liposomes which may be useful with the present embodiments include cationic liposomes, for example, as described in WO02/100435A1, U.S. Pat. No. 5,962,016, U.S. Application 2004/0208921, WO03/015757A1, WO04029213A2, U.S. Pat. Nos. 5,030,453, and 6,680,068, all of which are hereby incorporated by reference in their entirety without disclaimer.

In preparing such liposomes, any protocol described herein, or as would be known to one of ordinary skill in the art may be used. Additional non-limiting examples of preparing liposomes are described in U.S. Pat. Nos. 4,728,578, 4,728,575, 4,737,323, 4,533,254, 4,162,282, 4,310,505, and 4,921,706; International Applications PCT/US85/01161 and PCT/US89/05040, each incorporated herein by reference.

In certain embodiments, the lipid based nanoparticle is a neutral liposome (e.g., a DOPC liposome). “Neutral liposomes” or “non-charged liposomes”, as used herein, are defined as liposomes having one or more lipid components that yield an essentially-neutral, net charge (substantially non-charged). By “essentially neutral” or “essentially non-charged”, it is meant that few, if any, lipid components within a given population (e.g., a population of liposomes) include a charge that is not canceled by an opposite charge of another component (i.e., fewer than 10% of components include a non-canceled charge, more preferably fewer than 5%, and most preferably fewer than 1%). In certain embodiments, neutral liposomes may include mostly lipids and/or phospholipids that are themselves neutral under physiological conditions (i.e., at about pH 7).

Liposomes and/or lipid-based nanoparticles of the present embodiments may comprise a phospholipid. In certain embodiments, a single kind of phospholipid may be used in the creation of liposomes (e.g., a neutral phospholipid, such as DOPC, may be used to generate neutral liposomes). In other embodiments, more than one kind of phospholipid may be used to create liposomes. Phospholipids may be from natural or synthetic sources. Phospholipids include, for example, phosphatidylcholines, phosphatidylglycerols, and phosphatidylethanolamines; because phosphatidylethanolamines and phosphatidyl cholines are non-charged under physiological conditions (i.e., at about pH 7), these compounds may be particularly useful for generating neutral liposomes. In certain embodiments, the phospholipid DOPC is used to produce non-charged liposomes. In certain embodiments, a lipid that is not a phospholipid (e.g., a cholesterol) may be used

Phospholipids include glycerophospholipids and certain sphingolipids. Phospholipids include, but are not limited to, dioleoylphosphatidylycholine (“DOPC”), egg phosphatidylcholine (“EPC”), dilauryloylphosphatidylcholine (“DLPC”), dimyristoylphosphatidylcholine (“DMPC”), dipalmitoylphosphatidylcholine (“DPPC”), distearoylphosphatidylcholine (“DSPC”), 1-myristoyl-2-palmitoyl phosphatidylcholine (“MPPC”), 1-palmitoyl-2-myristoyl phosphatidylcholine (“PMPC”), 1-palmitoyl-2-stearoyl phosphatidylcholine (“PSPC”), 1-stearoyl-2-palmitoyl phosphatidylcholine (“SPPC”), dilauryloylphosphatidylglycerol (“DLPG”), dimyristoylphosphatidylglycerol (“DMPG”), dipalmitoylphosphatidylglycerol (“DPPG”), distearoylphosphatidylglycerol (“DSPG”), distearoyl sphingomyelin (“DSSP”), distearoylphophatidylethanolamine (“DSPE”), dioleoylphosphatidylglycerol (“DOPG”), dimyristoyl phosphatidic acid (“DMPA”), dipalmitoyl phosphatidic acid (“DPPA”), dimyristoyl phosphatidylethanolamine (“DMPE”), dipalmitoyl phosphatidylethanolamine (“DPPE”), dimyristoyl phosphatidylserine (“DMPS”), dipalmitoyl phosphatidylserine (“DPP S”), brain phosphatidylserine (“BPS”), brain sphingomyelin (“BSP”), dipalmitoyl sphingomyelin (“DPSP”), dimyristyl phosphatidylcholine (“DMPC”), 1,2-distearoyl-sn-glycero-3-phosphocholine (“DAPC”), 1,2-diarachidoyl-sn-glycero-3-phosphocholine (“DBPC”), 1,2-dieicosenoyl-sn-glycero-3-phosphocholine (“DEPC”), dioleoylphosphatidylethanolamine (“DOPE”), palmitoyloeoyl phosphatidylcholine (“POPC”), palmitoyloeoyl phosphatidylethanolamine (“POPE”), lysophosphatidylcholine, lysophosphatidylethanolamine, and dilinoleoylphosphatidylcholine.

B. Exosomes

The terms “microvesicle” and “exosomes,” as used herein, refer to a membranous particle having a diameter (or largest dimension where the particles is not spheroid) of between about 10 nm to about 5000 nm, more typically between 30 nm and 1000 nm, and most typically between about 50 nm and 750 nm, wherein at least part of the membrane of the exosomes is directly obtained from a cell. Most commonly, exosomes will have a size (average diameter) that is up to 5% of the size of the donor cell. Therefore, especially contemplated exosomes include those that are shed from a cell.

Exosomes may be detected in or isolated from any suitable sample type, such as, for example, body fluids. As used herein, the term “isolated” refers to separation out of its natural environment and is meant to include at least partial purification and may include substantial purification. As used herein, the term “sample” refers to any sample suitable for the methods provided by the present invention. The sample may be any sample that includes exosomes suitable for detection or isolation. Sources of samples include blood, bone marrow, pleural fluid, peritoneal fluid, cerebrospinal fluid, urine, saliva, amniotic fluid, malignant ascites, broncho-alveolar lavage fluid, synovial fluid, breast milk, sweat, tears, joint fluid, and bronchial washes. In one aspect, the sample is a blood sample, including, for example, whole blood or any fraction or component thereof. A blood sample suitable for use with the present invention may be extracted from any source known that includes blood cells or components thereof, such as venous, arterial, peripheral, tissue, cord, and the like. For example, a sample may be obtained and processed using well-known and routine clinical methods (e.g., procedures for drawing and processing whole blood). In one aspect, an exemplary sample may be peripheral blood drawn from a subject with cancer.

Exosomes may also be isolated from tissue samples, such as surgical samples, biopsy samples, tissues, feces, and cultured cells. When isolating exosomes from tissue sources it may be necessary to homogenize the tissue in order to obtain a single cell suspension followed by lysis of the cells to release the exosomes. When isolating exosomes from tissue samples it is important to select homogenization and lysis procedures that do not result in disruption of the exosomes. Exosomes contemplated herein are preferably isolated from body fluid in a physiologically acceptable solution, for example, buffered saline, growth medium, various aqueous medium, etc.

Exosomes may be isolated from freshly collected samples or from samples that have been stored frozen or refrigerated. In some embodiments, exosomes may be isolated from cell culture medium. Although not necessary, higher purity exosomes may be obtained if fluid samples are clarified before precipitation with a volume-excluding polymer, to remove any debris from the sample. Methods of clarification include centrifugation, ultracentrifugation, filtration, or ultrafiltration. Most typically, exosomes can be isolated by numerous methods well-known in the art. One preferred method is differential centrifugation from body fluids or cell culture supernatants. Exemplary methods for isolation of exosomes are described in (Losche et al., 2004; Mesri and Altieri, 1998; Morel et al., 2004). Alternatively, exosomes may also be isolated via flow cytometry as described in (Combes et al., 1997).

One accepted protocol for isolation of exosomes includes ultracentrifugation, often in combination with sucrose density gradients or sucrose cushions to float the relatively low-density exosomes. Isolation of exosomes by sequential differential centrifugations is complicated by the possibility of overlapping size distributions with other microvesicles or macromolecular complexes. Furthermore, centrifugation may provide insufficient means to separate vesicles based on their sizes. However, sequential centrifugations, when combined with sucrose gradient ultracentrifugation, can provide high enrichment of exosomes.

Isolation of exosomes based on size, using alternatives to the ultracentrifugation routes, is another option. Successful purification of exosomes using ultrafiltration procedures that are less time consuming than ultracentrifugation, and do not require use of special equipment have been reported. Similarly, a commercial kit is available (EXOMIR™, Bioo Scientific) which allows removal of cells, platelets, and cellular debris on one microfilter and capturing of vesicles bigger than 30 nm on a second microfilter using positive pressure to drive the fluid. However, for this process, the exosomes are not recovered, their RNA content is directly extracted from the material caught on the second microfilter, which can then be used for PCR analysis. HPLC-based protocols could potentially allow one to obtain highly pure exosomes, though these processes require dedicated equipment and are difficult to scale up. A significant problem is that both blood and cell culture media contain large numbers of nanoparticles (some non-vesicular) in the same size range as exosomes. For example, some miRNAs may be contained within extracellular protein complexes rather than exosomes; however, treatment with protease (e.g., proteinase K) can be performed to eliminate any possible contamination with “extraexosomal” protein.

### IV. PHARMACEUTICAL COMPOSITIONS

It is contemplated that exosomes that express or comprise a therapeutic protein, therapeutic antibody, inhibitory RNA, and/or small molecule drug can be administered systemically or locally to inhibit tumor cell growth and, most preferably, to sensitize the patient's cancer cells to immune checkpoint inhibitors. They can be administered intravenously, intrathecally, and/or intraperitoneally. They can be administered alone or in combination with anti-proliferative drugs. In one embodiment, they are administered in combination with immune checkpoint inhibitors to reduce the cancer load in the patient prior to surgery or other procedures. Alternatively, they can be administered in combination with immune checkpoint inhibitors after surgery to ensure that any remaining cancer (e.g., cancer that the surgery failed to eliminate) does not survive.

It is not intended that the present invention be limited by the particular nature of the therapeutic preparation. For example, such compositions can be provided in formulations together with physiologically tolerable liquid, gel, solid carriers, diluents, or excipients. These therapeutic preparations can be administered to mammals for veterinary use, such as with domestic animals, and clinical use in humans in a manner similar to other therapeutic agents. In general, the dosage required for therapeutic efficacy will vary according to the type of use and mode of administration, as well as the particular requirements of individual subjects.

Where clinical applications are contemplated, it may be necessary to prepare pharmaceutical compositions in a form appropriate for the intended application. Generally, pharmaceutical compositions may comprise an effective amount of one or more therapeutic agents dissolved or dispersed in a pharmaceutically acceptable carrier. The phrases “pharmaceutical or pharmacologically acceptable” refers to molecular entities and compositions that do not produce an adverse, allergic, or other untoward reaction when administered to an animal, such as, for example, a human, as appropriate. The preparation of a pharmaceutical composition will be known to those of skill in the art in light of the present disclosure, as exemplified by Remington's Pharmaceutical Sciences, 18th Ed., 1990, incorporated herein by reference. Moreover, for animal (e.g., human) administration, it will be understood that preparations should meet sterility, pyrogenicity, general safety, and purity standards as required by the FDA Office of Biological Standards.

Further in accordance with certain aspects of the present invention, the composition suitable for administration may be provided in a pharmaceutically acceptable carrier with or without an inert diluent. As used herein, “pharmaceutically acceptable carrier” includes any and all aqueous solvents (e.g., water, alcoholic/aqueous solutions, ethanol, saline solutions, parenteral vehicles, such as sodium chloride, Ringer's dextrose, etc.), non-aqueous solvents (e.g., fats, oils, polyol (for example, glycerol, propylene glycol, and liquid polyethylene glycol, and the like), vegetable oil, and injectable organic esters, such as ethyloleate), lipids, liposomes, dispersion media, coatings (e.g., lecithin), surfactants, antioxidants, preservatives (e.g., antibacterial or antifungal agents, anti-oxidants, chelating agents, inert gases, parabens (e.g., methylparabens, propylparabens), chlorobutanol, phenol, sorbic acid, thimerosal or combinations thereof), isotonic agents (e.g., sugars and sodium chloride), absorption delaying agents (e.g., aluminum monostearate and gelatin), salts, drugs, drug stabilizers, gels, resins, fillers, binders, excipients, disintegration agents, lubricants, sweetening agents, flavoring agents, dyes, fluid and nutrient replenishers, such like materials and combinations thereof, as would be known to one of ordinary skill in the art. The carrier should be assimilable and includes liquid, semi-solid, i.e., pastes, or solid carriers. In addition, if desired, the compositions may contain minor amounts of auxiliary substances, such as wetting or emulsifying agents, stabilizing agents, or pH buffering agents. The pH and exact concentration of the various components in a pharmaceutical composition are adjusted according to well-known parameters. The proper fluidity can be maintained, for example, by the use of a coating, such as lecithin, by the maintenance of the required particle size in the case of dispersion, and by the use of surfactants.

A pharmaceutically acceptable carrier is particularly formulated for administration to a human, although in certain embodiments it may be desirable to use a pharmaceutically acceptable carrier that is formulated for administration to a non-human animal but that would not be acceptable (e.g., due to governmental regulations) for administration to a human. Except insofar as any conventional carrier is incompatible with the active ingredient (e.g., detrimental to the recipient or to the therapeutic effectiveness of a composition contained therein), its use in the therapeutic or pharmaceutical compositions is contemplated. In accordance with certain aspects of the present invention, the composition is combined with the carrier in any convenient and practical manner, i.e., by solution, suspension, emulsification, admixture, encapsulation, absorption, and the like. Such procedures are routine for those skilled in the art.

The therapeutic agent can be formulated for parenteral administration, e.g., formulated for injection via the intravenous, intramuscular, sub-cutaneous, or even intraperitoneal routes. Typically, such compositions can be prepared as either liquid solutions or suspensions; solid forms suitable for use to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired. The actual dosage amount of a composition of the present invention administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (this such range includes intervening doses) or more per administration, and any range derivable therein. In non-limiting examples of a derivable range from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject.

The actual dosage amount of a composition administered to an animal patient can be determined by physical and physiological factors, such as body weight, severity of condition, the type of disease being treated, previous or concurrent therapeutic interventions, idiopathy of the patient, and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of a therapeutic agent. In other embodiments, an active compound may comprise between about 2% to about 75% of the weight of the unit, or between about 25% to about 60%, for example, and any range derivable therein. Naturally, the amount of active compound(s) in each therapeutically useful composition may be prepared in such a way that a suitable dosage will be obtained in any given unit dose of the compound. Factors, such as solubility, bioavailability, biological half-life, route of administration, product shelf life, as well as other pharmacological considerations, will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 milligram/kg/body weight or more per administration, and any range derivable therein. In non-limiting examples of a derivable range from the numbers listed herein, a range of about 5 milligram/kg/body weight to about 100 milligram/kg/body weight, about 5 microgram/kg/body weight to about 500 milligram/kg/body weight, etc., can be administered, based on the numbers described above.

### V. METHODS OF TREATMENT

The present invention provides methods of treating a cancer patient with a combination of a BMP7 inhibitor and an immune checkpoint blockade inhibitor. Such treatment may also be in combination with another therapeutic regime, such as chemotherapy. Certain aspects of the present invention can be used to select a cancer patient for treatment based on the presence of upregulated BMP7 expression in the patient's tumor, increased levels of BMP7 in the patient's blood or serum, increased levels of beta-catenin, Sox2, and/or PARP1 in the patient's tumor, decreased levels of CDKN2A, p38α, PTEN, PD-L1, YAP1_pS127, and/or granzyme B in the patient's tumor, or decreased levels of IL-1α, TNF-α, IFN-γ, and/or IL-2 in the patient's tumor infiltrating lymphocytes. In various aspects, about 5%, 10%, 15%, 20%, 25%, 30%, 35%, 40%, 45%, 50%, 55%, 60%, 65%, 70%, 75%, 80%, 85%, 90%, 95%, or 100% of the cells that comprise the cancer or the TILs may harbor an increase or decrease in one or more of the listed markers. Thus, in some aspects, about 5%, 10%, 15%, 20%, 25%, 30%, 35%, 40%, 45%, 50%, 55%, 60%, 65%, 70%, 75%, 80%, 85%, 90%, 95%, or 99% of the cells that comprise the cancer or the TILs may comprise normal levels of one or more of the listed markers. In other aspects, various percentages of cells comprising the cancer may harbor an altered expression level of one or more the listed markers. Other aspects of the present invention provide for selecting a cancer patient for treatment based on the patient having previously failed to respond to the administration of an immune checkpoint blockade inhibitor.

In some embodiments, the immunotherapy may be an immune checkpoint inhibitor. Immune checkpoints either turn up a signal (e.g., co-stimulatory molecules) or turn down a signal. Immune checkpoints either turn up a signal (e.g., co-stimulatory molecules) or turn down a signal. Immune checkpoint proteins that may be targeted by immune checkpoint blockade include adenosine A2A receptor (A2AR), B7-H3 (also known as CD276), B and T lymphocyte attenuator (BTLA), CCL5, CD27, CD38, CD8A, CMKLR1, cytotoxic T-lymphocyte-associated protein 4 (CTLA-4, also known as CD152), CXCL9, CXCR5, glucocorticoid-induced tumour necrosis factor receptor-related protein (GITR), HLA-DRB1, ICOS (also known as CD278), HLA-DQA1, HLA-E, indoleamine 2,3-dioxygenase 1 (IDO1), killer-cell immunoglobulin (KIR), lymphocyte activation gene-3 (LAG-3, also known as CD223), Mer tyrosine kinase (MerTK), NKG7, OX40 (also known as CD134), programmed death 1 (PD-1), programmed death-ligand 1 (PD-L1, also known as CD274), PDCD1LG2, PSMB10, STAT1, T cell immunoreceptor with Ig and ITIM domains (TIGIT), T-cell immunoglobulin domain and mucin domain 3 (TIM-3), V-domain Ig suppressor of T cell activation (VISTA, also known as C10orf54), and 4-1BB (CD137). In particular, the immune checkpoint inhibitors target the PD-1 axis and/or CTLA-4.

The immune checkpoint inhibitors may be drugs, such as small molecules, recombinant forms of ligand or receptors, or antibodies, such as human antibodies (e.g., International Patent Publication WO2015/016718; Pardoll, Nat Rev Cancer, 12(4): 252-264, 2012; both incorporated herein by reference). Known inhibitors of the immune checkpoint proteins or analogs thereof may be used, in particular chimerized, humanized, or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, a PD-1 binding antagonist is a molecule that inhibits the binding of PD-1 to its ligand binding partners. In a specific aspect, the PD-1 ligand binding partners are PD-L1 and/or PD-L2. In another embodiment, a PD-L1 binding antagonist is a molecule that inhibits the binding of PD-L1 to its binding partners. In a specific aspect, PD-L1 binding partners are PD-1 and/or B7-1. In another embodiment, a PD-L2 binding antagonist is a molecule that inhibits the binding of PD-L2 to its binding partners. In a specific aspect, a PD-L2 binding partner is PD-1. The antagonist may be an antibody, an antigen binding fragment thereof, an immunoadhesin, a fusion protein, or an oligopeptide. Exemplary antibodies are described in U.S. Pat. Nos. 8,735,553, 8,354,509, and 8,008,449, all of which are incorporated herein by reference. Other PD-1 axis antagonists for use in the methods provided herein are known in the art, such as described in U.S. Patent Application Publication Nos. 2014/0294898, 2014/022021, and 2011/0008369, all of which are incorporated herein by reference.

In some embodiments, a PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342.

Another immune checkpoint protein that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA-4 is similar to the T-cell co-stimulatory protein, CD28, and both molecules bind to CD80 and CD86, also called B7-1 and B7-2 respectively, on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide. Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in U.S. Pat. No. 8,119,129; PCT Publn. Nos. WO 01/14424, WO 98/42752, WO 00/37504 (CP675,206, also known as tremelimumab; formerly ticilimumab); U.S. Pat. No. 6,207,156; Hurwitz et al. (1998) Proc Natl Acad Sci USA, 95(17): 10067-10071; Camacho et al. (2004) J Clin Oncology, 22(145): Abstract No. 2505 (antibody CP-675206); and Mokyr et al. (1998) Cancer Res, 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2, and CDR3 domains of the VL region of ipilimumab. In another embodiment, the antibody competes for binding with and/or binds to the same epitope on CTLA-4 as the above-mentioned antibodies. In another embodiment, the antibody has an at least about 90% variable region amino acid sequence identity with the above-mentioned antibodies (e.g., at least about 90%, 95%, or 99% variable region identity with ipilimumab). Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

Another immune checkpoint protein that can be targeted in the methods provided herein is lymphocyte-activation gene 3 (LAG-3), also known as CD223. The complete protein sequence of human LAG-3 has the Genbank accession number NP-002277. LAG-3 is found on the surface of activated T cells, natural killer cells, B cells, and plasmacytoid dendritic cells. LAG-3 acts as an “off” switch when bound to MHC class II on the surface of antigen-presenting cells. Inhibition of LAG-3 both activates effector T cells and inhibitor regulatory T cells. In some embodiments, the immune checkpoint inhibitor is an anti-LAG-3 antibody (e.g., a human antibody, a humanized antibody, or a chimeric antibody), an antigen binding fragment thereof, an immunoadhesin, a fusion protein, or oligopeptide. Anti-human-LAG-3 antibodies (or VH and/or VL domains derived therefrom) suitable for use in the present methods can be generated using methods well known in the art. Alternatively, art recognized anti-LAG-3 antibodies can be used. An exemplary anti-LAG-3 antibody is relatlimab (also known as BMS-986016) or antigen binding fragments and variants thereof (see, e.g., WO 2015/116539). Other exemplary anti-LAG-3 antibodies include TSR-033 (see, e.g., WO 2018/201096), MK-4280, and REGN3767. MGD013 is an anti-LAG-3/PD-1 bispecific antibody described in WO 2017/019846. FS118 is an anti-LAG-3/PD-L1 bispecific antibody described in WO 2017/220569.

Another immune checkpoint protein that can be targeted in the methods provided herein is V-domain Ig suppressor of T cell activation (VISTA), also known as C10orf54. The complete protein sequence of human VISTA has the Genbank accession number NP_071436. VISTA is found on white blood cells and inhibits T cell effector function. In some embodiments, the immune checkpoint inhibitor is an anti-VISTA3 antibody (e.g., a human antibody, a humanized antibody, or a chimeric antibody), an antigen binding fragment thereof, an immunoadhesin, a fusion protein, or oligopeptide. Anti-human-VISTA antibodies (or VH and/or VL domains derived therefrom) suitable for use in the present methods can be generated using methods well known in the art. Alternatively, art recognized anti-VISTA antibodies can be used. An exemplary anti-VISTA antibody is JNJ-61610588 (also known as onvatilimab) (see, e.g., WO 2015/097536, WO 2016/207717, WO 2017/137830, WO 2017/175058). VISTA can also be inhibited with the small molecule CA-170, which selectively targets both PD-L1 and VISTA (see, e.g., WO 2015/033299, WO 2015/033301).

Another immune checkpoint protein that can be targeted in the methods provided herein is indoleamine 2,3-dioxygenase (IDO). The complete protein sequence of human IDO has Genbank accession number NP_002155. In some embodiments, the immune checkpoint inhibitor is a small molecule IDO inhibitor. Exemplary small molecules include BMS-986205, epacadostat (INCB24360), and navoximod (GDC-0919).

Another immune checkpoint protein that can be targeted in the methods provided herein is CD38. The complete protein sequence of human CD38 has Genbank accession number NP_001766. In some embodiments, the immune checkpoint inhibitor is an anti-CD38 antibody (e.g., a human antibody, a humanized antibody, or a chimeric antibody), an antigen binding fragment thereof, an immunoadhesin, a fusion protein, or oligopeptide. Anti-human-CD38 antibodies (or VH and/or VL domains derived therefrom) suitable for use in the present methods can be generated using methods well known in the art. Alternatively, art recognized anti-CD38 antibodies can be used. An exemplary anti-CD38 antibody is daratumumab (see, e.g., U.S. Pat. No. 7,829,673).

Another immune checkpoint protein that can be targeted in the methods provided herein is ICOS, also known as CD278. The complete protein sequence of human ICOS has Genbank accession number NP_036224. In some embodiments, the immune checkpoint inhibitor is an anti-ICOS antibody (e.g., a human antibody, a humanized antibody, or a chimeric antibody), an antigen binding fragment thereof, an immunoadhesin, a fusion protein, or oligopeptide. Anti-human-ICOS antibodies (or VH and/or VL domains derived therefrom) suitable for use in the present methods can be generated using methods well known in the art. Alternatively, art recognized anti-ICOS antibodies can be used. Exemplary anti-ICOS antibodies include JTX-2011 (see, e.g., WO 2016/154177, WO 2018/187191) and GSK3359609 (see, e.g., WO 2016/059602).

Another immune checkpoint protein that can be targeted in the methods provided herein is T cell immunoreceptor with Ig and ITIM domains (TIGIT). The complete protein sequence of human TIGIT has Genbank accession number NP_776160. In some embodiments, the immune checkpoint inhibitor is an anti-TIGIT antibody (e.g., a human antibody, a humanized antibody, or a chimeric antibody), an antigen binding fragment thereof, an immunoadhesin, a fusion protein, or oligopeptide. Anti-human-TIGIT antibodies (or VH and/or VL domains derived therefrom) suitable for use in the present methods can be generated using methods well known in the art. Alternatively, art recognized anti-TIGIT antibodies can be used. An exemplary anti-TIGIT antibody is MK-7684 (see, e.g., WO 2017/030823, WO 2016/028656).

Another immune checkpoint protein that can be targeted in the methods provided herein is OX40, also known as CD134. The complete protein sequence of human OX40 has Genbank accession number NP_003318. In some embodiments, the immune checkpoint inhibitor is an anti-OX40 antibody (e.g., a human antibody, a humanized antibody, or a chimeric antibody), an antigen binding fragment thereof, an immunoadhesin, a fusion protein, or oligopeptide. Anti-human-OX40 antibodies (or VH and/or VL domains derived therefrom) suitable for use in the present methods can be generated using methods well known in the art. Alternatively, art recognized anti-OX40 antibodies can be used. An exemplary anti-OX40 antibody is PF-04518600 (see, e.g., WO 2017/130076). ATOR-1015 is a bispecific antibody targeting CTLA4 and OX40 (see, e.g., WO 2017/182672, WO 2018/091740, WO 2018/202649, WO 2018/002339).

Another immune checkpoint protein that can be targeted in the methods provided herein is glucocorticoid-induced tumour necrosis factor receptor-related protein (GITR), also known as TNFRSF18 and AITR. The complete protein sequence of human GITR has Genbank accession number NP_004186. In some embodiments, the immune checkpoint inhibitor is an anti-GITR antibody (e.g., a human antibody, a humanized antibody, or a chimeric antibody), an antigen binding fragment thereof, an immunoadhesin, a fusion protein, or oligopeptide. Anti-human-GITR antibodies (or VH and/or VL domains derived therefrom) suitable for use in the present methods can be generated using methods well known in the art. Alternatively, art recognized anti-GITR antibodies can be used. An exemplary anti-GITR antibody is TRX518 (see, e.g., WO 2006/105021).

Another immune checkpoint protein that can be targeted in the methods provided herein is T-cell immunoglobulin and mucin-domain containing-3 (TIM3), also known as HAVCR2. The complete protein sequence of human TIM3 has Genbank accession number NP_116171. In some embodiments, the immune checkpoint inhibitor is an anti-TIM3 antibody (e.g., a human antibody, a humanized antibody, or a chimeric antibody), an antigen binding fragment thereof, an immunoadhesin, a fusion protein, or oligopeptide. Anti-human-TIM3 antibodies (or VH and/or VL domains derived therefrom) suitable for use in the present methods can be generated using methods well known in the art. Alternatively, art recognized anti-TIM3 antibodies can be used. Exemplary anti-TIM3 antibodies include LY3321367 (see, e.g., WO 2018/039020), MBG453 (see, e.g., WO 2015/117002) and TSR-022 (see, e.g., WO 2018/085469).

Another immune checkpoint protein that can be targeted in the methods provided herein is 4-1BB, also known as CD137, TNFRSF9, and ILA. The complete protein sequence of human 4-1BB has Genbank accession number NP_001552. In some embodiments, the immune checkpoint inhibitor is an anti-4-1BB antibody (e.g., a human antibody, a humanized antibody, or a chimeric antibody), an antigen binding fragment thereof, an immunoadhesin, a fusion protein, or oligopeptide. Anti-human-4-1BB antibodies (or VH and/or VL domains derived therefrom) suitable for use in the present methods can be generated using methods well known in the art. Alternatively, art recognized anti-4-1BB antibodies can be used. An exemplary anti-4-1BB antibody is PF-05082566 (utomilumab; see, e.g., WO 2012/032433).

The term “subject” or “patient” as used herein refers to any individual to which the subject methods are performed. Generally, the patient is human, although as will be appreciated by those in the art, the patient may be an animal. Thus, other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of patient.

“Treatment” and “treating” refer to administration or application of a therapeutic agent to a subject or performance of a procedure or modality on a subject for the purpose of obtaining a therapeutic benefit of a disease or health-related condition. For example, a treatment may include administration chemotherapy, immunotherapy, radiotherapy, performance of surgery, or any combination thereof.

The methods described herein are useful in treating cancer. Generally, the terms “cancer” and “cancerous” refer to or describe the physiological condition in mammals that is typically characterized by unregulated cell growth. More specifically, cancers that are treated in connection with the methods provided herein include, but are not limited to, solid tumors, metastatic cancers, or non-metastatic cancers. In certain embodiments, the cancer may originate in the lung, kidney, bladder, blood, bone, bone marrow, brain, breast, colon, esophagus, duodenum, small intestine, large intestine, colon, rectum, anus, gum, head, liver, nasopharynx, neck, ovary, pancreas, prostate, skin, stomach, testis, tongue, or uterus.

The cancer may specifically be of the following histological type, though it is not limited to these: neoplasm, malignant; carcinoma; non-small cell lung cancer; renal cancer; renal cell carcinoma; clear cell renal cell carcinoma; lymphoma; blastoma; sarcoma; carcinoma, undifferentiated; meningioma; brain cancer; oropharyngeal cancer; nasopharyngeal cancer; biliary cancer; pheochromocytoma; pancreatic islet cell cancer; Li-Fraumeni tumor; thyroid cancer; parathyroid cancer; pituitary tumor; adrenal gland tumor; osteogenic sarcoma tumor; neuroendocrine tumor; breast cancer; lung cancer; head and neck cancer; prostate cancer; esophageal cancer; tracheal cancer; liver cancer; bladder cancer; stomach cancer; pancreatic cancer; ovarian cancer; uterine cancer; cervical cancer; testicular cancer; colon cancer; rectal cancer; skin cancer; giant and spindle cell carcinoma; small cell carcinoma; small cell lung cancer; papillary carcinoma; oral cancer; oropharyngeal cancer; nasopharyngeal cancer; respiratory cancer; urogenital cancer; squamous cell carcinoma; lymphoepithelial carcinoma; basal cell carcinoma; pilomatrix carcinoma; transitional cell carcinoma; papillary transitional cell carcinoma; adenocarcinoma; gastrointestinal cancer; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma with squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; lentigo maligna melanoma; acral lentiginous melanoma; nodular melanoma; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; an endocrine or neuroendocrine cancer or hematopoietic cancer; pinealoma, malignant; chordoma; central or peripheral nervous system tissue cancer; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; B-cell lymphoma; malignant lymphoma; Hodgkin's disease; Hodgkin's; low grade/follicular non-Hodgkin's lymphoma; paragranuloma; malignant lymphoma, small lymphocytic; malignant lymphoma, large cell, diffuse; malignant lymphoma, follicular; mycosis fungoides; mantle cell lymphoma; Waldenstrom's macroglobulinemia; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; chronic lymphocytic leukemia (CLL); acute lymphoblastic leukemia (ALL); Hairy cell leukemia; chronic myeloblastic leukemia; and hairy cell leukemia.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the invasiveness of a tumor, reduction in the growth rate of the cancer, or prevention of metastasis. Treatment of cancer may also refer to prolonging survival of a subject with cancer.

Likewise, an effective response of a patient or a patient's “responsiveness” to treatment refers to the clinical or therapeutic benefit imparted to a patient at risk for, or suffering from, a disease or disorder. Such benefit may include cellular or biological responses, a complete response, a partial response, a stable disease (without progression or relapse), or a response with a later relapse. For example, an effective response can be reduced tumor size or progression-free survival in a patient diagnosed with cancer.

Regarding neoplastic condition treatment, depending on the stage of the neoplastic condition, neoplastic condition treatment involves one or a combination of the following therapies: surgery to remove the neoplastic tissue, radiation therapy, and chemotherapy. Other therapeutic regimens may be combined with the administration of the anticancer agents, e.g., therapeutic compositions and chemotherapeutic agents. For example, the patient to be treated with such anti-cancer agents may also receive radiation therapy and/or may undergo surgery.

In the case of non-small cell lung cancer, the patient may undergo surgery to remove cancerous tissue. Such surgery may be a pneumonectomy, lobectomy, segmentectomy, wedge resection, or sleeve resection. The patient may undergo radiation treatment, such as external beam radiation therapy or brachytherapy. The patient may undergo radiofrequency ablation, which uses high-energy radio waves to heat the tumor and destroy cancer cells. The patient may undergo chemotherapy with one or more of cisplatin, carboplatin, paclitaxel, albumin-bound paclitaxel, docetaxel, gemcitabine, vinorelbine, irinotecan, etoposide, vinblastine, and pemetrexed. In addition, bevacizumab, ramucirumab, or necitumuab may also be used. If the patient's cancer expresses an increased level of EGFR, then the patient may also be treated with erlotinib, afatinib, gefitinib, osimertinib, or dacomitinib. If the patient's cancer has an ALK gene rearrangement, then the patient may also be treated with crizotinib, ceritinib, alectinib, brigatinib, or lorlatinib. If the patient's cancer expresses an altered BRAF protein, then the patient may also be treated with dabrafenib or trametinib.

For the treatment of disease, the appropriate dosage of a therapeutic composition will depend on the type of disease to be treated, as defined above, the severity and course of the disease, previous therapy, the patient's clinical history and response to the agent, and the discretion of the physician. The agent may be suitably administered to the patient at one time or over a series of treatments.

A. Combination Treatments

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. A tissue, tumor, or cell can be contacted with one or more compositions or pharmacological formulation(s) comprising one or more of the agents or by contacting the tissue, tumor, and/or cell with two or more distinct compositions or formulations. Also, it is contemplated that such a combination therapy can be used in conjunction with radiotherapy, surgical therapy, or immunotherapy.

Administration in combination can include simultaneous administration of two or more agents in the same dosage form, simultaneous administration in separate dosage forms, and separate administration. That is, the subject therapeutic composition and another therapeutic agent can be formulated together in the same dosage form and administered simultaneously. Alternatively, subject therapeutic composition and another therapeutic agent can be simultaneously administered, wherein both the agents are present in separate formulations. In another alternative, the therapeutic agent can be administered just followed by the other therapeutic agent or vice versa. In the separate administration protocol, the subject therapeutic composition and another therapeutic agent may be administered a few minutes apart, or a few hours apart, or a few days apart.

An anti-cancer first treatment may be administered before, during, after, or in various combinations relative to a second anti-cancer treatment. The administrations may be in intervals ranging from concurrently to minutes to days to weeks. In embodiments where the first treatment is provided to a patient separately from the second treatment, one would generally ensure that a significant period of time did not expire between the time of each delivery, such that the two compounds would still be able to exert an advantageously combined effect on the patient. In such instances, it is contemplated that one may provide a patient with the first therapy and the second therapy within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below a combination of a BMP7 inhibitor and an immune checkpoint inhibitor is “A” and another anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present invention to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as folinic acid; aceglatone; aldophosphamide glycoside; aminolevulinic acid; eniluracil; amsacrine; bestrabucil; bisantrene; edatraxate; defofamine; demecolcine; diaziquone; elformi thine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; PARP inhibitors, such as olaparib, rucaparib, niraparib, talazoparib, BMN673, iniparib, CEP 9722, or ABT888 (veliparab); CDK4/6 inhibitors, such as ribociclib, palbociclib, ademaciclib, or trilaciclib; androgen inhibitor and anti-androgens, such as cyproterone acetate, megestrol acetate, chlormadinone acetate, spironolactone, oxendolone, osaterone acetate, flutamide, bicalutamide, nilutamide, topilutamide, enzalutamide, apalutamide, dienogest, drospirenone, medrogestone, nomegestrol acetate, promegestone, trimegeston, ketoconazole, abiraterone acetate, seviteronel, aminoglutethimide, finasteride, dutasteride, epristeride, alfatradiol, saw palmetto extract (Serenoa repens), medrogestone, and bifluranol; carboplatin, procarbazine,plicomycin, gemcitabien, navelbine, farnesyl-protein tansferase inhibitors, transplatinum, and pharmaceutically acceptable salts, acids, or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that additional immunotherapies may be used in combination or in conjunction with methods of the invention. In the context of cancer treatment, immunotherapeutics, generally, rely on the use of immune effector cells and molecules to target and destroy cancer cells. Rituximab (Rituxan®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, Infection Immun., 66(11):5329-5336, 1998; Christodoulides et al., Microbiology, 144(Pt 11):3027-3037, 1998); cytokine therapy, e.g., interferons α, β, and γ, IL-1, GM-CSF, and TNF (Bukowski et al., Clinical Cancer Res., 4(10):2337-2347, 1998; Davidson et al., J. Immunother., 21(5):389-398, 1998; Hellstrand et al., Acta Oncologica, 37(4):347-353, 1998); gene therapy, e.g., TNF, IL-1, IL-2, and p53 (Qin et al., Proc. Natl. Acad. Sci. USA, 95(24):14411-14416, 1998; Austin-Ward and Villaseca, Revista Medica de Chile, 126(7):838-845, 1998; U.S. Pat. Nos. 5,830,880 and 5,846,945); and monoclonal antibodies, e.g., anti-CD20, anti-ganglioside GM2, and anti-p185 (Hanibuchi et al., Int. J. Cancer, 78(4):480-485, 1998; U.S. Pat. No. 5,824,311). It is contemplated that one or more anti-cancer therapies may be employed with the antibody therapies described herein.

In some embodiment, the immune therapy could be adoptive immunotherapy, which involves the transfer of autologous antigen-specific T cells generated ex vivo. The T cells used for adoptive immunotherapy can be generated either by expansion of antigen-specific T cells or redirection of T cells through genetic engineering. Isolation and transfer of tumor specific T cells has been shown to be successful in treating melanoma. Novel specificities in T cells have been successfully generated through the genetic transfer of transgenic T cell receptors or chimeric antigen receptors (CARs). CARS are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. CARs have successfully allowed T cells to be redirected against antigens expressed at the surface of tumor cells from various malignancies including lymphomas and solid tumors.

In one embodiment, the present application provides for a combination therapy for the treatment of cancer wherein the combination therapy comprises adoptive T cell therapy and a checkpoint inhibitor. In one aspect, the adoptive T cell therapy comprises autologous and/or allogenic T-cells. In another aspect, the autologous and/or allogenic T-cells are targeted against tumor antigens.

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present invention, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present invention to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present invention to improve the treatment efficacy.

### VI. KITS AND DIAGNOSTICS

In various aspects of the invention, a kit is envisioned containing, diagnostic agents, therapeutic agents and/or delivery agents. In some embodiments, the kit may comprise reagents for assessing a patient selection marker, such as BMP7 expression levels, in a patient sample. In some embodiments, the present invention contemplates a kit for preparing and/or administering a therapy of the invention. The kit may comprise reagents capable of use in administering an active or effective agent(s) of the invention. Reagents of the kit may include one or more anti-cancer component of a combination therapy, as well as reagents to prepare, formulate, and/or administer the components of the invention or perform one or more steps of the inventive methods.

In some embodiments, the kit may also comprise a suitable container means, which is a container that will not react with components of the kit, such as an eppendorf tube, an assay plate, a syringe, a bottle, or a tube. The container may be made from sterilizable materials such as plastic or glass. The kit may further include an instruction sheet that outlines the procedural steps of the methods and will follow substantially the same procedures as described herein or are known to those of ordinary skill.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Materials & Methods

Patient tumor and blood samples. Tumor biopsies from three patients with disease progression after pembrolizumab (NCT02444741) or ipilimumab (NCT02239900) were examined. Paraffin-embedded tissues were used for the IHC analysis. Biopsies were collected prior treatment with radiotherapy. Pretreatment blood samples from patients with PD on pembrolizumab (NCT02444741; NCT02402920) (n=5) versus patients with PR or SD (n=4) were collected in EDTA tubes. Blood samples were centrifuged at 1,000×g for 10 min, and plasma samples were collected and kept at −80° C. until analysis. All analyses were approved by the UT MD Anderson Cancer Center institutional review board (protocols 2014-1020 and 2013-0882).

Cell lines. The 344SQ parental cell line (344SQP) was a generous gift from Dr. Jonathan Kurie (MD Anderson). From the 344SQP cell line, we generated an anti-PD1-resistant cell line (344SQR) as previously described (Wang et al., 2016). RAW 264.7 and EL4 cell lines were obtained from the American Type Culture Collection (ATCC; Manassas, Va., USA). Cell lines were cultured in complete medium (RPMI-1640 supplemented with 100 units/mL penicillin, 100 μg/mL streptomycin, and 10% heat-inactivated fetal bovine serum) and incubated at 37° C. in 5% CO₂. Cell lines were validated by DDC Medical (available at ddcmedical.com; Fairfield, Ohio) by short-tandem-repeat (STR) DNA fingerprinting.

Establishment of stable BMP7- and p38α-knockdown cells. To establish stable BMP7-knockdown cells, GIPZ Non-silencing Lentiviral shRNA Control (Catalog #RHS4348, Dharmacon) and specific mouse shRNA targeting BMP7 (pGIPZ Clone ID V2LMM_12865, Dharmacon) and p38a (pGIPZ Clone ID V3LMM_415230, Dharmacon) viral supernatants were purchased from the shRNA and ORFeome Core at MD Anderson Cancer Center. 344SQR and EL4 cells were infected and incubated with the viral particles supplemented with Polybrene (8 μg/mL, Sigma) overnight at 37° C. Puromycin (1 μg/mL) was used to select and maintain BMP7-knockdown in 344SQR cells and p38α-knockdown in EL4 cells. Stable repression was verified by qPCR and western blotting.

In vivo studies. All mouse studies were approved by the Institutional Animal Care and Use Committee (IACUC) of The University of Texas MD Anderson Cancer Center before their initiation; animal care was provided according to IACUC standards, and all mice had been bred and were maintained in our own specific pathogen-free mouse colony. For RRBS, RPPA and TILs studies, primary tumors were established by subcutaneous injection of 344SQP or 344SQR cells (0.5×10⁶ in 100 μL of sterile phosphate buffered saline [PBS]) into the leg of syngeneic 129Sv/Ev mice (female, 12-16 weeks old). Mice were then given intraperitoneal injections of anti-PD1 or control IgG antibodies (10 mg/kg) (Bio X cell), starting on day 4 after tumor cell inoculation and continuing twice per week for a total of 4 doses. At 24 hours after the last anti-PD1 treatment, tumor tissues were collected for DNA (2 mice/group) and protein isolation (2 or 3 mice/group). For TILs isolation, tumor tissues (3 mice/group) were collected a week after the last treatment with anti-PD1. For tumor growth and survival studies, primary tumors were established by subcutaneous injection of 344SQR ctrl or 344SQR shBMP7 cells (0.5×10⁶ in 100 μL of sterile PBS into the leg of syngeneic 129Sv/Ev mice (female, 12-16 weeks old). The mice were then given intraperitoneal injections of anti-PD-1, anti-PDL1 (Durvalumab, Pharmacy MD Anderson), anti-CTLA4 or control IgG antibodies (10 mg/kg) (Bio X cell), starting on day 4 after tumor cell inoculation and continuing twice per week for a total of 4 doses. Lastly, primary tumors were established by subcutaneous injection of 344SQR cells (0.5×10⁶ in 100 μL of sterile PBS) into the leg of syngeneic 129Sv/Ev mice (female, 12-16 weeks old), which were then given intraperitoneal injections of anti-PD1 (10 mg/kg), control IgG antibodies (10 mg/kg), follistatin (R&D Systems, Catalog #769-FS-025) (0.1 mg/kg) or follistatin (0.1 mg/Kg) plus anti-PD1 (10 mg/kg), starting on day 4 after tumor cell inoculation. Anti-PD1 antibody was given twice per week for a total of 4 doses; follistatin was given four times per week before and after anti-PD1 for a total of 8 doses. Tumors were measured with calipers three times per week and recorded as tumor volume (in mm³)=width²×length/2. Mice were euthanized when tumors became ulcerated or reached a maximum size of 1500 mm³. A two-way analysis of variance was done to compare tumor growth curves between groups. Mouse survival rates were analyzed by using the Kaplan-Meier method and compared with log-rank tests.

Tissue processing and flow cytometry. Tumor cells or TILs were blocked with FcR blocker for 10 min at room temperature. T-effectors (CD3, CD4, CD8) and Macrophages and MDSCs (Gr-1, CD11b, F4/80, CD206) were stained at room temperature for 30 min. All antibodies were purchased from Biolegend. For intracellular staining of IFN-γ, cells were fixed and permeabilized according to the manufacturer's instructions (Biolegend) and stained with anti-IFN-γ. All samples were analyzed with an LSRII flow cytometer and FlowJo software (version 10.0.7).

Isolation of tumor-infiltrating immune cells. Freshly isolated primary tumor tissues (from 2 or 3 mice/group) were washed with ice-cold PBS and digested with 250 μg/mL of Liberase TR (Roche) and 20 μg/mL DNase I (Roche) and incubated for 45 minutes at 37° C. with shaking. Fetal bovine serum was added, and samples were filtered followed by Histopaque-1077 (Sigma-Aldrich) gradient isolation of TILs. The TILs in the interphase were collected and washed with PBS plus 2% fetal bovine serum. TILs were used for Nanostring, qPCR, or flow cytometry analysis.

NanoString immune profiling. RNA samples from TILs isolated from 344SQP (n=2) or 344SQR (n=3) tumors treated with anti-PD1 as previously described were isolated with Triazol (Life Technologies) according to the manufacturer's protocol. Samples with 100 ng of RNA were submitted for NanoString analysis using the PanCancer immune profiling panel of 770 genes at the Genomic and RNA Profiling Core at Baylor College of Medicine. The analysis was done in R (version 3.5.1). The Reporter Code Count data received from the core were preprocessed with the NanoStringNorm package. Genes expressed at different levels between groups were identified by a P value, obtained from the moderated t statistic from the LIMMA package, of <0.05. To support visual data exploration, a heatmap for the most significant cases was generated with the heatmap.2 function from the gplots package.

Bio-Plex mouse cytokine 23-plex assay. Serum samples were collected from mice bearing 344SQP or 344SQR tumors treated with anti-PD1 twice per week for a total of 4 doses as follows. At 24 hours after the last anti-PD1 treatment, whole blood samples were collected by cardiac puncture and centrifuged at 1,000×g for 10 min, and serum was collected and kept in −80° C. until analysis. Serum was diluted 1:4 with diluent solutions from the BioPlex Multiplex assay (BioRad). Twenty-three cytokines, including IL-1α, IL-1β, TNF-α, RANTES, IFN-γ, and IL-2 were measured by ELISA according to the manufacturer's protocol (Biorad, Catalog #m60009rdpd).

Protein extraction and Western blot analysis. Total protein was extracted by using NP40 lysis buffer (0.5% NP40, 250 mmol/1 NaCl, 50 mmol/1 HEPES, 5 mmol/1 ethylenediaminetetraacetic acid, and 0.5 mmol/1 egtazic acid) supplemented with protease inhibitors cocktails (Sigma-Aldrich). Lysates were centrifuged at 10,000×g for 10 minutes, and the supernatant was collected for experiments. Protein lysates (40 μg) were resolved on denaturing gels with 4-20% sodium dodecyl sulfate-polyacrylamide and transferred to nitrocellulose membranes (Bio-Rad Laboratories, Hercules, Calif.). Membranes were probed with primary antibodies directed against BMP7 (Abcam, Catalog #ab56023), Phospho-Smad1 (Ser463/465)/Smad5 (Ser463/465)/Smad9 (Ser465/467) (Cell Signaling Technologies, Catalog #13820), p38 MAPK (Cell Signaling Technologies, Catalog #8690), Vinculin (Cell Signaling Technologies, Catalog #13901), β-Actin (Cell Signaling Technologies, Catalog #3700), and a secondary antibody conjugated with horseradish peroxidase (Amersham GE Healthcare). The secondary antibody was visualized by using a chemiluminescent reagent (Pierce ECL kit, Thermo Fisher Scientific, Waltham, Mass., USA).

Reduced representation bisulfite sequencing (RRBS). RRBS was done by the Epigenomics Profiling Core and Science Park NGS facility at MD Anderson Cancer Center. A KAPA Library Quantification Kit (KAPA Biosystems) was utilized to quantify RRBS libraries for pooling, and a final concentration of 1.5 nM was loaded onto an Illumina cBOT for cluster generation before sequencing on an Illumina HiSeq3000 using a Single Read 50 bp run. The libraries were sequenced using 50 bases single-read protocol on Illumina HiSeq 3000 instrument. 49-85 million reads were generated per sample. Mapping: The adapters were removed from 3′ ends of the reads by Trim Galore! (version 0.4.1) (available on the world wide web at bioinformatics.babraham.ac.uk/projects/trim_galore/) and cutadapt (version 1.9.1). Then the reads were mapped to mouse genome mm10 by the bisulfite converted read mapper Bismark (version v0.16.1) and Bowtie (version 1.1.2). 92-94% reads were mapped to the mouse genome, with 66-68% uniquely mapped. 33-56 million uniquely mapped reads were used in the final analysis. Methylation Calling: The methylation percentages for CpG sites were calculated by the bismark_methylation_extractor script from Bismark and an in-house Perl script. Differential Methylation: the differential methylation on CpG sites was statistically assessed by R/Bioconductor package methylKit (version 0.9.5). The CpG sites with read coverage ≥20 in all the samples were qualified for the test. The significance of differential methylation on gene level was calculated using Stouffer's zscore method by combining all the qualified CpG sites inside each gene's promoter region (defined as −1000 bp to +500 of TSS), and was corrected to FDR by Benjamini & Hochberg (BH) method. Heatmap and clustering: heatmap and clustering were performed on the top 10 hypermethylated genes and top 10 hypomethylated genes (by FDR) from the genes with number of CpG sites in promoter ≥5 and methylation difference ≥20%. Hierarchical clustering was done by hclust function in R using the average methylation percentage of all the qualified CpG sites in each gene's promoter region. Before clustering, for each gene, the methylation percentages across samples were centered by median and rescaled so that the sum of the squares is 1.0. Euclidean distance and ward.D2 clustering method were used for the clustering of the genes. The heatmap was plotted by heatmap.2 function in R.

Pyrosequencing Methylation Assay (PMA). Bisulfite PCR was done by the Epigenomics Profiling Core Facility at MD Anderson Cancer Center as described previously (Kroeger et al., 2008). Briefly, genomic DNA (2 μg) was denatured with 0.2 M NaOH at 37° C. for 10 min followed by incubation with 30 μL of freshly prepared 10 mM hydroquinone and 520 μL of 3 M sodium bisulfite (pH 5.0) at 50° C. for 16 h. DNA was purified on a Wizard Miniprep Column (Promega), desulfonated with 0.3 M NaOH at 25° C. for 5 min, precipitated with ammonium acetate and ethanol, and dissolved in 50 μL of Tris-EDTA buffer (10 mM Tris-HCl, 1 mM EDTA, pH 8.0). Bisulfite-treated DNA (40-80 ng) was amplified with gene-specific primers in a two-step PCR. PCR products from the second step were cloned into the pCR4-TOPO vector (Invitrogen), transformed into competent bacteria, and sequenced.

Quantitative polymerase chain reaction (qPCR). Total RNA was isolated from cells and tumors with Triazol (Life Technologies) according to the manufacturer's protocol. For studies of BMP7, p38α, IL-1α, IL-1β, TNF-α, RANTES, IL-2, and IFN-γ expression, mRNA was retrotranscribed with the iScript™ gDNA Clear cDNA Synthesis Kit (BioRad) and analyzed by qPCR using SYBR Green (Life Technologies) with specific primers according to the manufacturer's protocol. The comparative Ct method was used to calculate the relative abundance of mRNAs compared with ACTB (beta-actin) expression for cancer cells or CD45 expression for immune cells.

Enzyme-linked immunosorbent assay (ELISA). Serum was collected from mice bearing 344SQP or 344SQR tumors treated with anti-PD1 twice per week for a total of 4 doses. A week after the last anti-PD1 treatment, whole blood samples were collected by cardiac puncture and centrifuged at 1,000 xg for 10 min, and serum was collected and kept at −80° C. until analysis. Culture supernatants were freshly collected from 344SQP, 344SQP, 344SQR ctrl, and shBMP7 tumors and directly submitted for analysis. Plasma samples from patients with PD on pembrolizumab (NCT02444741; NCT02402920) and radiotherapy versus patients with PR or SD were collected as previously described. BMP7 levels in serum, plasma, or culture supernatants were measured by ELISA according to the manufacturer's protocol (ThermoFisher Scientific, Catalog #EHBMP7 and EMBMP7).

Reverse phase protein array. Tissues were homogenized with a sonicator in a solution containing complete protease and PhosSTOP phosphatase inhibitor cocktail tablets (Roche Applied Science, Mannheim, Germany), 1 mM Na₃VO₄ and lysis buffer (1% Triton X-100, 50 mM HEPES [pH 7.4], 150 mM NaCl, 1.5 mM MgCl₂, 1 mM EGTA, 100 mM NaF, 10 mM NaPPi, 10% glycerol, 1 mM phenylmethylsulfonyl fluoride [serine protease inhibitor], and 10 μg/mL aprotinin) Samples were vortexed frequently on ice and then centrifuged. Cleared supernatants were collected and proteins quantified with a BCA kit (Pierce Biotechnology, Inc., Rockford, Ill.). Tumor lysates were serially diluted two-fold for 5 dilutions (from undiluted to 1:16 dilution) and arrayed on nitrocellulose-coated slides in an 11×11 format. Samples were probed with 243 antibodies with a tyramide-based signal amplification approach and visualized by DAB colorimetric reaction. RPPA analyses were done by the RPPA-Functional Proteomics core facility at MD Anderson Cancer Center as previously described (Li et al., 2013). The analysis was done in R (version 3.5.1). Normalized data were first log 2-transformed (log 2(x+1)). Proteins expressed at different levels between groups were identified by a P value (obtained from the moderated t statistic from the LIMMA package) of <0.05. To support visual data exploration, a heatmap for the most significant cases was generated by using the heatmap.2 function from the gplots package.

Immunohistochemical analysis (IHC). Formalin-fixed patient samples and mouse tissues were processed in an automatic tissue processor, embedded in paraffin (Peloris, Leica) and cut into 4-μm sections. IHC staining was done in an automated staining system (Leica Bond Max, Leica Microsystems, Vista, Calif., USA). Briefly, slides were deparaffinized and hydrated, and antigen was retrieved by incubating in citrate buffer, pH 6.0, for 1 hour with BMP7 (Abcam, Catalog #ab56023), p38a (Thermo Fisher Scientific, Catalog #PA5-17713), SMAD1(Thermo Fisher Scientific, Catalog #38-5400), anti-Phospho-SMAD1/SMADS (Ser463, Ser465) (Thermo Scientific-Life Technologies, Catalog #MA5-15124), anti-mannose receptor (CD206) (Abcam, Catalog #ab64693), or anti-CD4 (Bioss, Catalog #bs-0647R) according to the manufacturer's protocol. Slides were examined with a Leica DMI6000B microscope (Leica, Buffalo Grove, Ill.), and images were captured by a charge-coupled device camera and imported into the Advanced Spot Image analysis software package.

Immunofluorescence analysis. RAW 264.7 cells were counted with a hemocytometer (0.4% Trypan blue solution), diluted to 200,000, and seeded in 4-well culture slides (Lab-Tek, Catalog #154917), and allowed to attach overnight. Cells were treated with 250 ng BMP7 (R&D Systems, Catalog #5666-BP-010) or follistatin (R&D Systems, Catalog #769-FS-025) and incubated for 24 h, and then fixed with 1% paraformaldehyde for 10 minutes, followed by a 10-minute wash in 70% ethanol at room temperature. Cells were then treated with 0.1% NP40 in PBS for 20 minutes, washed in PBS four times, and then blocked with 5% bovine serum albumin in PBS for 30 minutes. Cells were then incubated with p38a MAPK (L53F8) (Cell Signaling, Catalog #9228) and Phospho-Smad1 (Ser463/465), Smad5 (Ser463/465), and Smad9 (Ser465/467) (D5B10) (Cell Signaling, Catalog #13820) in 5% bovine serum albumin in PBS overnight according to the manufacturer's protocol. The next day, cells were incubated with anti-rabbit IgG (H+L), F(ab′)2 Fragment (Alexa Fluor 488 Conjugate) (Cell Signaling, Catalog #4412), or Anti-mouse IgG (H+L), F(ab′)2 Fragment (Alexa Fluor 488 Conjugate) (Cell Signaling, Catalog #4408) secondary antibody according to the manufacturer's protocol. Then, cells were incubated in the dark with 4 4,6-diamidino-2-phenylindole dihydrochloride (1 mg/mL) in PBS for 5 minutes, and coverslips were mounted on a slide with an antifade solution (Molecular Probes; Invitrogen, Waltham, Mass.). Slides were examined with a fluorescence microscope (Leica, Buffalo Grove, Ill.), and images were captured by a charge-coupled device camera and imported into the Advanced Spot Image analysis software package.

Co-culture experiments and treatments. Viable cells were counted with a hemocytometer (0.4% Trypan blue solution) and diluted to 40,000 cells per well in 24-wells plates. Cells from 344SQP, 344SQR, 344SQ ctrl, or 344SQ-shBMP7 tumors were seeded at the top inserts (24-mm Transwell with 0.4-μm pore polycarbonate membrane insert, Sigma-Aldrich), and RAW 264.7 or CD4⁺ T cells were seeded at the bottom of the transwell system. CD4⁺ T cells were isolated from splenocytes by using Dynabeads Untouched Mouse CD4 Cells Kit (Thermo Fisher Scientific—Life Technologies, Catalog #11416D) and activated with LEAF purified anti-mouse CD3c antibody (5 μg/mL) and LEAF purified anti-mouse CD28 antibody (1 μg/mL) (Biolegend). Cells were then cultured in complete medium (RPMI-1640 supplemented with 100 units/mL penicillin, 100 μg/mL streptomycin, and 10% heat-inactivated fetal bovine serum) and incubated at 37° C. in 5% CO₂ for 24 or 48 hours, after which cells were treated with 250 ng of BMP7 (R&D Systems, Catalog #5666-BP-010) or follistatin (R&D Systems, Catalog #769-FS-025) for 24 or 48 hours. RNA was then isolated from RAW 264.7 or CD4⁺ T cells and analyzed for p38α, IL-1α, IL-1β, TNF-α, RANTES, IL-2 and IFN-γ expression with qPCR.

Transfection siRNA. siRNA targeting p38a (Life Technologies, Cat #4390771) and its respective negative-control negative control (Life Technologies, Cat #4390843) were reverse-transfected into RAW 264.7 macrophages with Lipofectamine 2000 (Life Technologies) to a final concentration of 100 nm/L.

Flow cytometry. TILs were then blocked with anti-CD16/CD32 before being stained for flow cytometry. For flow cytometry purposes, fluorochrome-conjugated anti-CD3 (Cat #100353), anti-CD4 (Cat #100406), anti-CD8 (Cat #100734), anti-CD45 (Cat #103126), anti-CD11b (Cat #101226), anti-CD11c (Cat #117310), anti-F4/80 (Cat #123108), and anti-CD206 (Cat #141716) antibodies were purchased from BioLegend. Samples were analyzed with an LSR II flow cytometer and FlowJo software.

mRNA Correlation for TCGA samples. Correlation analysis for mRNA data for TCGA Lung Adenocacinoma cohort was performed in R (version 3.4.1; available on the world wide web at r-project.org/). For this cohort of patients, RNASeqv-2 quantification mRNA-seq data from the TCGA portal for gene expression was retrieved. Log 2-transformation was applied to mRNA-seq data. The Spearman's rank-order correlation test was applied to measure the strength of the association between different pairs of mRNA expression levels in tumor samples.

Statistical analysis. Prism 8.0 software (GraphPad) was used for statistical analyses; the methods used are stated in the figure legends. Statistical significance was accepted at P≤0.05. Student's t tests were used to compare differences between individual groups, and tumor growth curves were compared with two-way analysis of variance, with error bars representing the standard deviation (s.d.).

### Example 2—BMP7 is Upregulated in Tumors that Did not Respond to Anti-PD1 Therapy

The interaction between PD1 and its ligand programmed cell death 1 ligand 1 (PDL1) inhibits T-cell proliferation, survival, and effector functions, which in turn limit antigen-specific T-cell responses and antitumor immunity (Topalian et al., 2012a). Antibodies blocking PD1/PDL1 have led to impressive clinical responses in some patients with melanoma, lung cancer, or renal cell carcinoma; however, the objective response rates to single-agent anti-PD1 or PDL1 therapies are only 15%-25% in chemotherapy-refractory non-small cell lung cancer (NSCLC) (Brahmer et al., 2012; Topalian et al., 2012b; Gettinger & Herbt, 2014). That many patients either do not respond to or develop recurrence after immunotherapy indicates the presence of intrinsic and/or acquired resistance (Kelderman et al., 2014). This observation raises fundamental questions about other mechanisms underlying nonresponse and potential strategies to overcome anti-PD1/PDL1 resistance—a major unmet therapeutic need. To answer these questions, an anti-PD1-resistant preclinical tumor model was generated involving an anti-PD1 variant of the murine lung cancer cell line 344SQ in syngeneic mice (Wang et al., 2016). In seeking to identify mechanisms of resistance to anti-PD1 therapy, several promising pathways were found that could be used as both biomarkers and therapeutic targets to overcome immune evasion. Specifically, anti-PD1-resistant tumors presented overexpression of the bone morphogenetic protein (BMP)-7, also known as OP-1. BMP7 is a secreted protein that belongs to the transforming growth factor β (TGF-β) superfamily and regulates proliferation, differentiation, and apoptosis in many different cell types by altering target gene transcription. BMPs ligands bind and form heteromeric complexes with two types of serine/threonine kinase receptors (type I and type II) on the cell surface which then activates the phosphorylation and gene expression of “small mothers against decapentaplegic” (SMADs) proteins in cells (Kretzschmar et al., 1997a; Liu et al., 1996; Kretzschmar et al., 1997b).

BMP7 and other BMPs have been shown to also signal via mitogen-activated protein kinase 14 (p38α) in a dose-dependent manner (Hu et al., 2004; Lee et al., 2002; Iwasaki et al., 1999; Awazu et al., 2017; Wang et al., 2016; Takahashi et al., 2008). P38 appears to play a critical role in regulation of the expression of a number of proinflammatory chemokines and cytokines induced by IFN-λ (Valledor et al., 2008). In macrophages, p38a is activated by LPS and TLR4, which subsequently activates proinflammatory cytokines, including IL-1 and TNF-α (Lee et al., 1994; Kim et al., 2004; Zhu et al., 2000; Baldassare et al., 1999). BMPs can act both as tumor suppressors or oncogenes depending on the cellular context and tumor type. BMP7 has been reported in a wide range of human cancers and has been associated with metastasis and poor prognosis (Aoki et al., 2011; Motoyama et al., 2008; Megumi et al., 2012; Alarmo et al., 2007; Alarmo et al., 2006; Rothhammer et al., 2005). In lung cancer, BMP7 overexpression was associated with lymph node involvement and an indicator of bone metastasis (Chen et al., 2010; Liu et al., 2012). Unlike TGFβ, the immunoregulatory functions of BMPs are not as well understood. Nonetheless, accumulating studies have shown that BMPs also regulate immune cell responses and are immunosuppressive in the setting of cancer (Chen & Ten Dijke, 2016). For example, BMPs have been shown to regulate activation, growth, and cytokine secretion in macrophages and promote PDL1 and PDL2 upregulation in dendritic cells (DCs) (Hong et al., 2009; Kwon et al., 2009; Lee et al., 2013; Martinez et al., 2011). Treatment with BMP7 in vitro and in vivo significantly enhanced monocyte polarization into M2-macrophages (Rocher et al., 2012; Singla et al., 2016; Rocher & Singla, 2013).

BMP7 is overexpressed in anti-PD1 resistant mouse model and in NSCLC patients that progressed on anti-PD1 therapy. This suggests that BMP7 regulates pro-inflammatory responses in the tumor microenvironment (TME) by suppressing p38 signaling in macrophages and CD4⁺ T cells. Furthermore, BMP7 inhibition in combination with anti-PD1 activates CD4⁺ and CD8⁺ T cells in tumors, decreases M2 macrophages, and resensitizes tumors to immunotherapies.

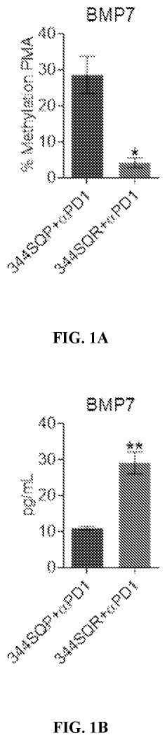

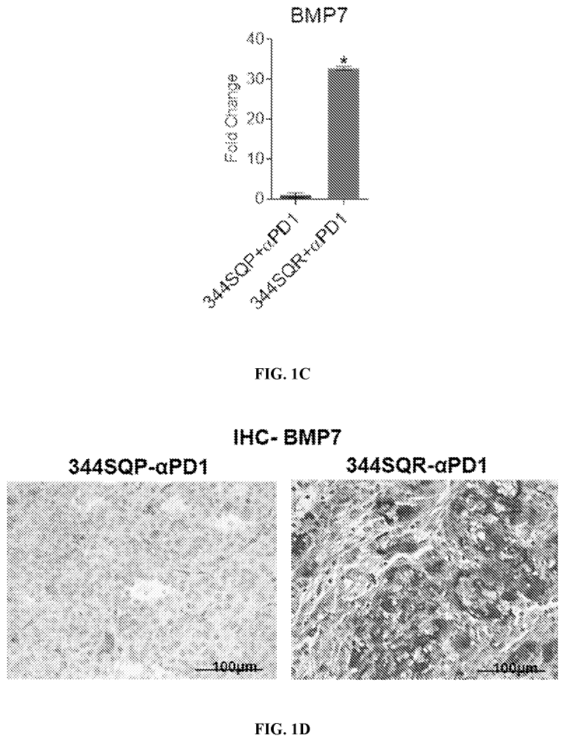

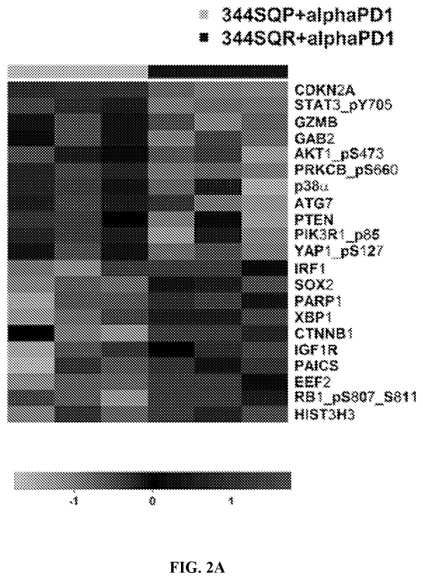

A preclinical NSCLC model (p53^{R172HΔg}/+K-ras^{LA1/+}) with acquired resistance to anti-PD1 was previously generated in a syngeneic host repeatedly dosed with anti-mouse PD1 antibodies (Wang et al., 2016). Here, methylation differences in specific genomic regions were investigated by comparing anti-PD1-resistant tumors (344SQR) with their parental-tumor counterparts (344SQP) using reduced-representation bisulfite sequencing. Overall, genes were hypomethylated in anti-PD1-resistant tumors compared with parental tumors, as assessed by the percentage of CpG sites methylated. Although some genes such as KCNK4, RAVER2, DMRTA1, TMEM200b, RAX, CLIC6, RAB42, NEIL2, PALM3, and NAV1 were hypermethylated, others including BMP7, SNORD37, KLHL1, FAM196a, AMPD3, PAMR1, NLGN3, AGTR1b, KIF21a, and SLC2a13 were hypomethylated in 344SQR tumors compared with parental tumors. It was confirmed that the BMP7 promoter CpG is hypomethylated, with an average of 4.28% in 344SQR tumors versus 28.68% in 344SQP tumors (FIG. 1A).

Global profiling using microarray analysis identified BMP7 as one of the top genes upregulated in the anti-PD1-resistant model (344SQR) compared to parental tumors (344SQP), which led to focusing on validating BMP7 as a target for resistance to anti-PD1. Because BMP7 is secreted, BMP7 levels were evaluated in plasma from mice bearing resistant and parental tumors. BMP7 levels were higher in serum from mice bearing 344SQR tumors than in mice with parental tumors (FIG. 1B). In addition, BMP7 upregulation at the mRNA and protein levels in 344SQR and 344SQP tumors treated with anti-PD1 therapy was validated by qPCR and immunohystochemical staining (IHC) (FIGS. 1C,1D). Thus, tumors resistant to anti-PD1 therapy have upregulated BMP7 expression and secretion via promoter hypomethylation, and BMP7 overexpression may promote resistance to immunotherapies.

### Example 3—BMP7 Modulates p38a in Anti-PD1-Resistant Tumors and Immune Cells in the Tumor Microenvironment

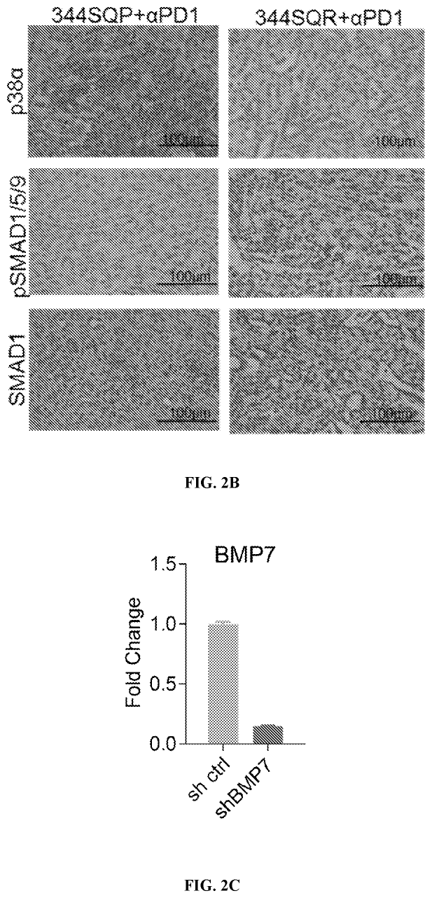

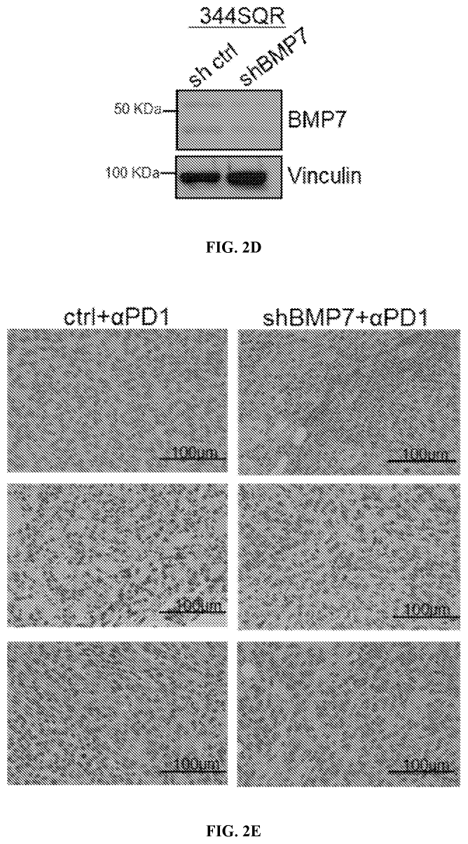

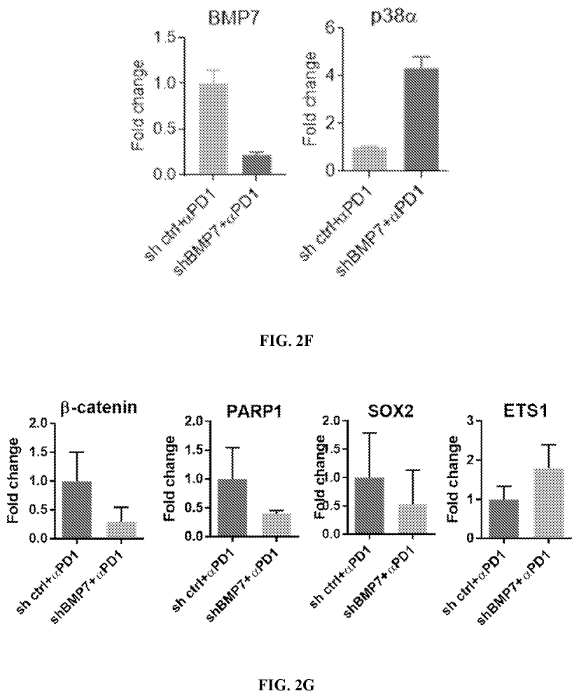

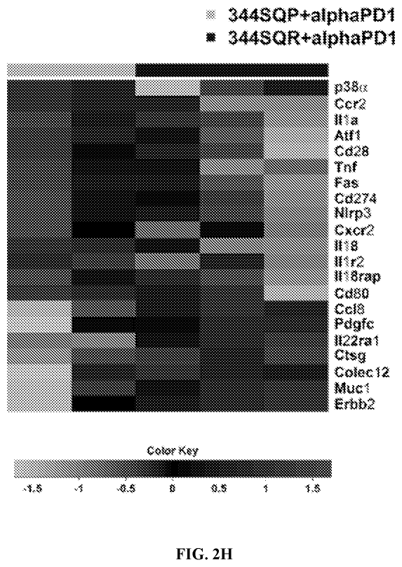

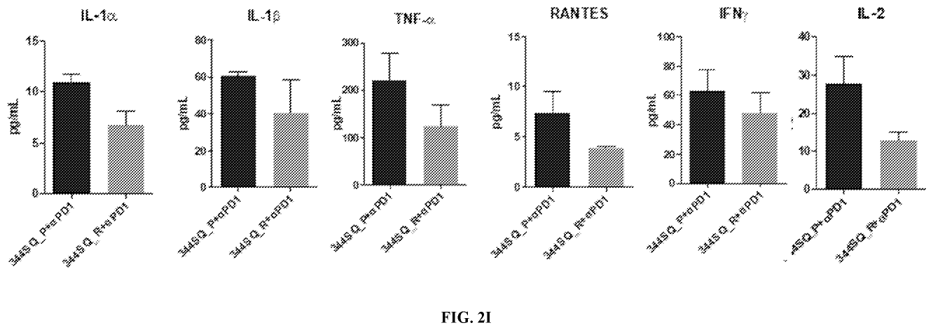

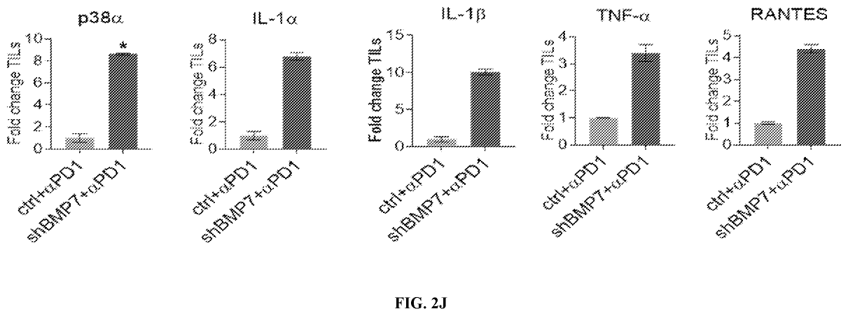

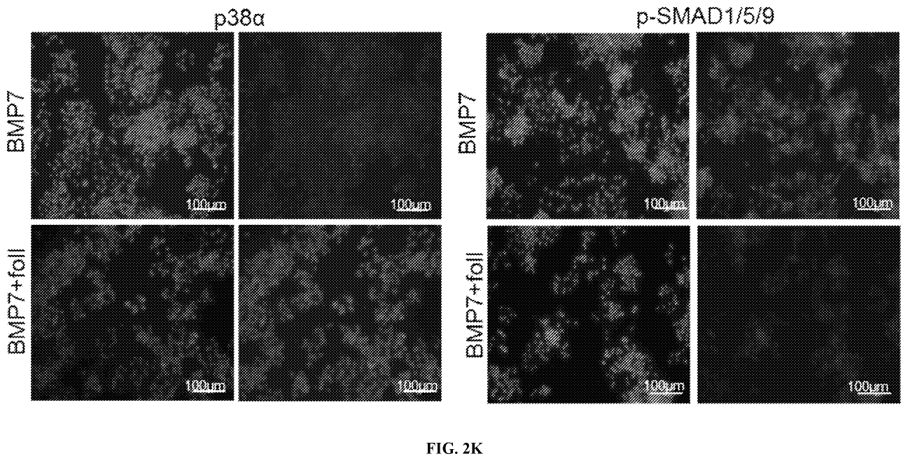

To identify the molecular mechanism by which BMP7 upregulation promotes resistance to anti-PD1, the expression levels and activation status of 243 proteins in 344SQP and 344SQR tumors treated with anti-PD1 were analyzed. Proteins known to be modulated by BMP7 were found to be expressed at different levels in resistant tumors than in parental tumors. For example, p38a was downregulated and CTNNB1 (β-catenin) was upregulated in 344SQR tumors compared to 344SQP tumors treated with anti-PD1 (FIG. 2A). Other downregulated proteins included CDKN2A (p16), CD274 (PD-L1), PDK1, AIM1, STAT3_pY705, ATG7, YAP1_pS127, PTEN, and granzyme B (GZMB), and other upregulated proteins included IGF1R, HIST3H3, SOX2, XBP1, YBX1, PARP1, and RB1_pS807_S811 (FIG. 2A). Because p38a is inhibited by BMP7 (Takahashi et al., 2008; Li et al., 2015) via SMAD1 at high BMP7 concentrations (Hu et al., 2004), the activation status of p38α, SMAD1, and SMAD1/5/9 in 344SQP tumors versus 344SQR tumors was analyzed. 344SQR tumors treated with anti-PD1 expressed less p38a and had higher activation of SMAD1 than did parental tumors (FIG. 2B).

Next, whether p38a downregulation was dependent on BMP7 was evaluated. To do so, 344SQR stable cell lines overexpressing shRNAs against BMP7 were first established (FIGS. 2C,2D). p38a levels were upregulated in BMP7-knockdown tumors treated with anti-PD1 relative to control tumors. Then, the activation status of p38α, SMAD1, and SMAD1/5/9 in BMP7-knockdown tumors treated with anti-PD1 and control tumors were evaluated. p38a mRNA and protein levels were found to be upregulated in BMP7-knockdown tumors than control tumors treated with anti-PD1, and SMAD1 activation was lower in BMP7-knockdown tumors than control tumors treated with anti-PD1 (FIGS. 2E,2F). These data suggest that BMP7 downregulates p38a via SMAD1 in tumors resistant to anti-PD1 therapy. The expression of proteins previously correlated with BMP7 was also evaluated and several were found to be upregulated in 344SQR tumors compared to parental tumors, including Beta Catenin, PARP1, SOX2, and ETS1. These proteins were downregulated in BMP7-knockdown tumors compared to parental tumors (FIG. 2G).

Next, it was hypothesized that secreted BMP7 negatively affects immune cells in the tumor microenvironment of anti-PD1-resistant tumors. Tumor-infiltrating lymphocytes (TILs) collected from 344SQP and 344SQR tumors treated with anti-PD1 were analyzed using the Nanostring Immune Panel. MUC1, ERBB2, and COLEC12 were found to be upregulated in TILs from resistant tumors compared to parental (FIG. 2H). Strikingly, p38a was downregulated in TILs from 344SQR tumors relative to parental tumors (FIG. 2H). Interestingly, different expression levels of Slc7a11, CD274 (PDL1), NLRP3, and MUC1 were found in TILs isolated from 344SQR versus parental tumors treated with anti-PD1 (FIG. 2H). Several inflammatory cytokines and genes regulated by p38a were downregulated in TILs from the 344SQR tumors relative to parental tumors, including IL-1α, IL-1β, TNF-α, and ATF1 (FIG. 2H). To validate these findings, serum levels of p38α-regulated cytokines and chemokines from mice bearing 344SQR or 344SQP tumors were analyzed. In agreement with the Nanostring data, levels of IL-1α, IL-1β, and TNF-α were downregulated in serum from mice bearing 344SQR tumors versus parental tumors (FIG. 2I). CCL5 (RANTES), IFN-γ, and IL-2 (also related to p38a signaling) were also downregulated in serum from mice bearing 344SQR tumors versus parental tumors, although those findings were not evident in the Nanostring data (FIG. 2I). IL-12p70, IL-2p40, and KC were also downregulated in serum from mice bearing 344SQR tumors versus parental tumors. No significant changes were found for other cytokines analyzed such as IL-6, IL-10 and MCP-1. In order to determine if p38a downregulation in TILs was dependent on BMP7 secretion in the tumor microenvironment (TME), the expression levels of p38α, IL1-α, IL1-β, TNF-α, CCL5 (RANTES), IFN-γ, and IL-2 were evaluated in TILs isolated from BMP7 knockdown tumors treated with anti-PD1 compared to control. p38α, IL1-α, IL1-β, TNF-α, CCL5, IFN-γ, and IL-2 expression levels were increased on TILs isolated from BMP7 knockdown tumors compared to control (FIG. 2J). Next, whether BMP7 promotes p38a downregulation via SMAD1 in TILs, as was previously seen for 344SQR tumors, was evaluated. p38a expression was higher in macrophage cell line (RAW 264.7) treated with BMP7 plus follistatin compared with BMP7 alone (FIG. 2K). On the other hand, SMAD1/5/9 activation was lower in cells treated with BMP7 plus follistatin versus BMP7 alone (FIG. 2K). These results suggest that BMP7 regulates p38a expression via SMAD1 signaling not only in tumors resistant to anti-PD1 but also in TILs isolated from these tumors relative to control.

### Example 4—BMP7 Reduced Macrophage-Mediated Pro-Inflammatory Signaling Via p38α

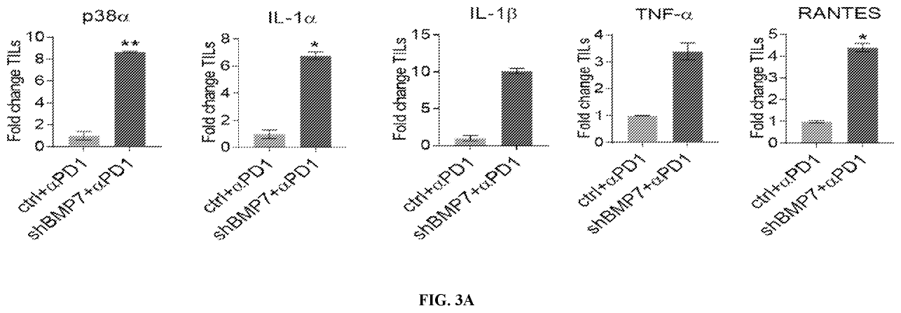

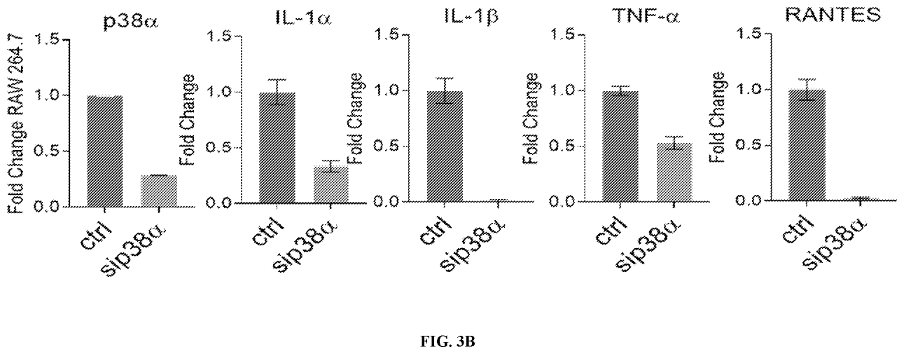

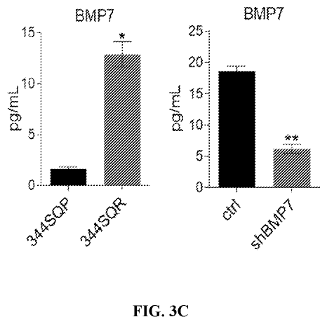

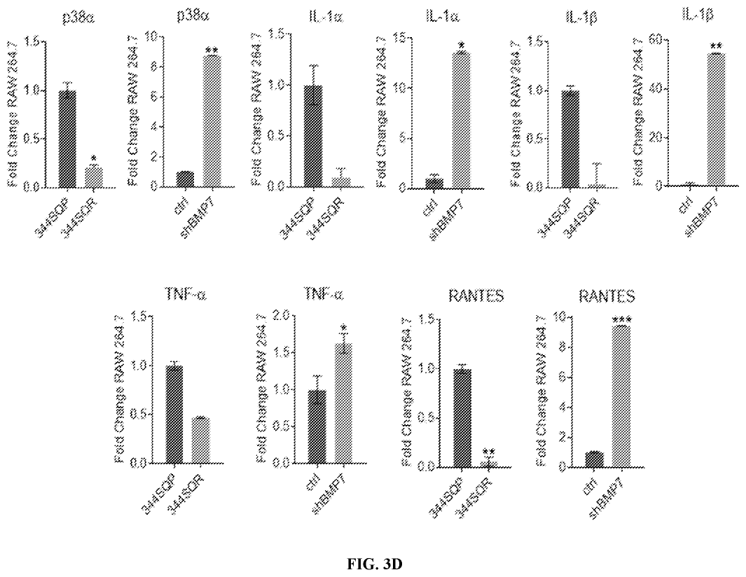

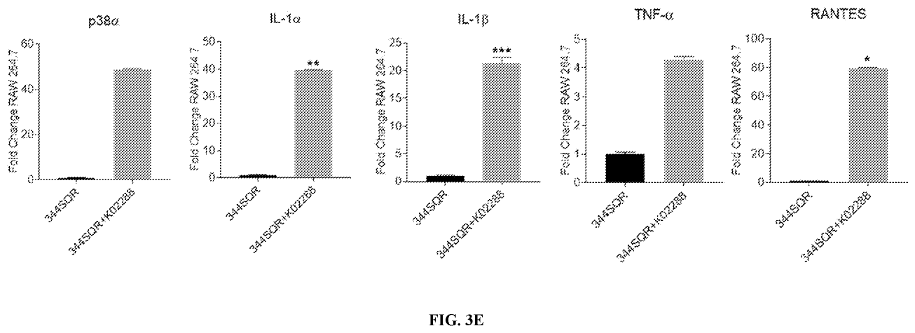

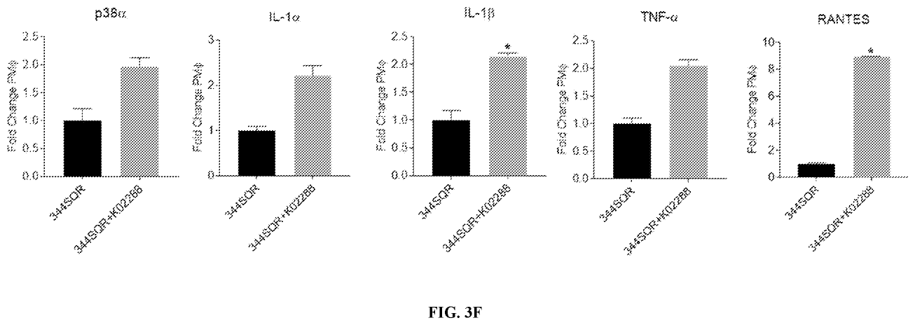

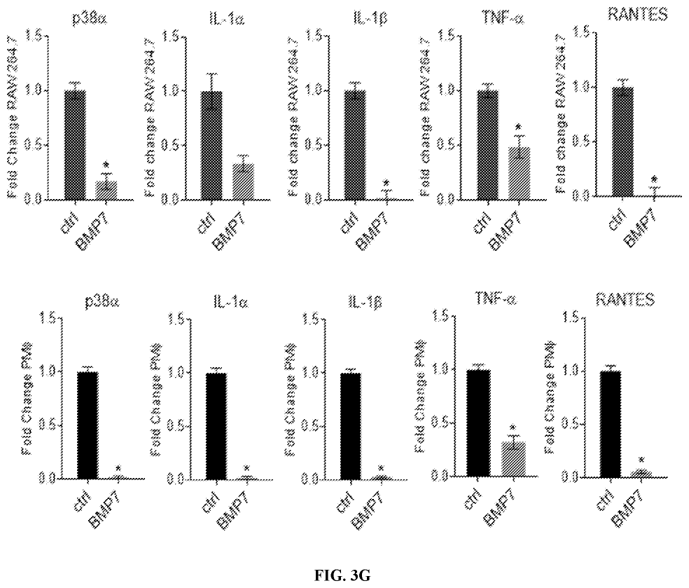

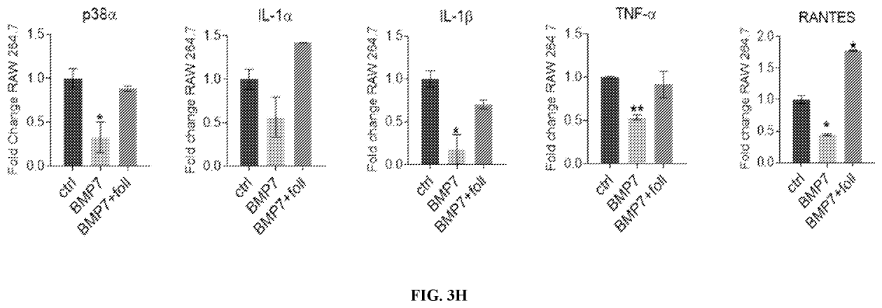

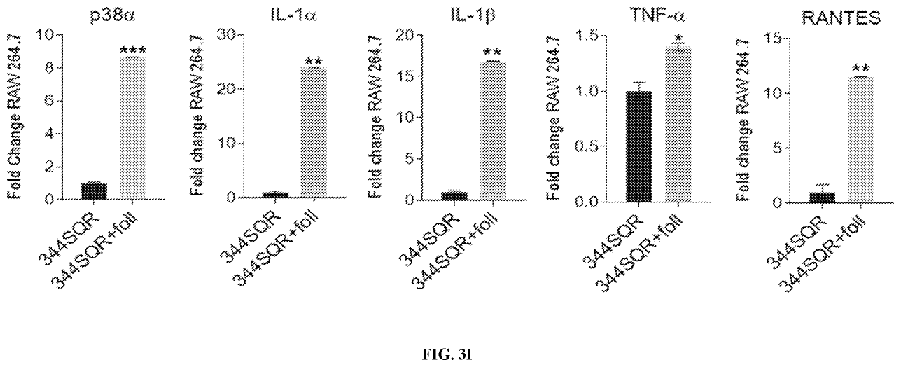

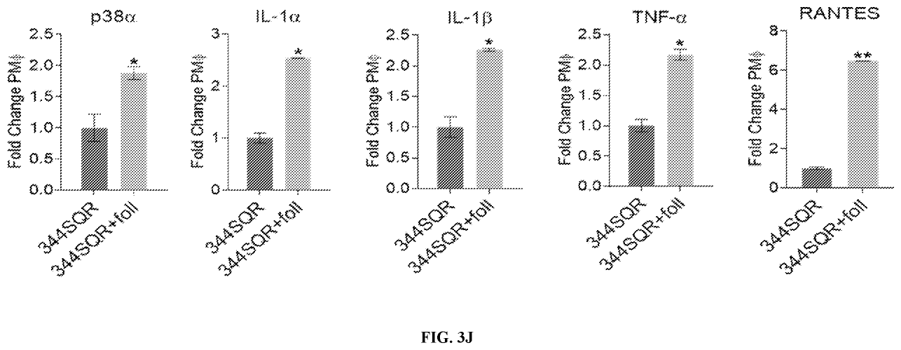

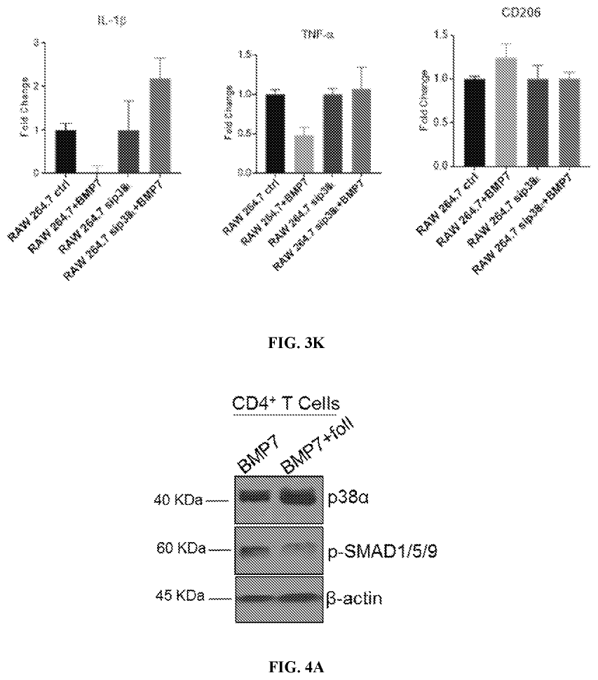

In order to determine if p38a downregulation in TILs depended on BMP7 secretion in the tumor microenvironment, the expression of p38a and p38α-regulated cytokines and chemokines in TILs isolated from BMP7-knockdown tumors as compared with control tumors treated with anti-PD1 was evaluated. p38α, IL-1α, IL-1β, TNF-α, and RANTES expression levels were increased in TILs from BMP7-knockdown tumors versus control (FIG. 3A). p38a was then silenced in RAW 264.7 cells with siRNAs and IL-1α, IL-1β, TNF-α, and RANTES expression analyzed. As expected, silencing p38a decreased the expression of p38α-regulated cytokines and chemokines in RAW 264.7 cells (FIG. 3B).

Next, whether tumor-secreted BMP7 regulates IL-1α, IL-1β, TNF-α, and RANTES via p38a in macrophages was investigated. BMP7 levels were measured in media from 344SQP vs. 344SQR, and 344SQR ctrl vs. 344SQR-shBMP7. As expected, 344SQR cells secreted higher levels of BMP7 than 344SQP cells, and BMP7-knockdown cells secreted lower BMP7 levels than 344SQR ctrl (FIG. 3C). Then, RAW 264.7 cells were co-cultured with 344SQP or 344SQR, and 344SQR shBMP7 or 344SQR ctrl. Macrophages cultured with 344SQR cells had lower expression of p38a and p38α-regulated cytokines and chemokines compared with cells co-cultured with 344SQP cells (FIG. 3D). On the other hand, macrophages co-cultured with 344SQR-shBMP7 cells had higher expression of p38α and p38α-regulated cytokines and chemokines compared with 344SQR ctrl (FIG. 3D).

Next, whether a BMP receptor inhibitor K02288 downregulates the expression of IL-1α, IL-1β, TNF-α, and RANTES in macrophages was investigated. 344SQR were seeded at the top inserts, and RAW 264.7 cells or peritoneal macrophages were seeded at the bottom of the transwell system. Cells were then cultured in complete medium (RPMI-1640 supplemented with 100 units/mL penicillin, 100 μg/mL streptomycin, and 10% heat-inactivated fetal bovine serum) and incubated at 37° C. in 5% CO₂ for 24 or 48 hours, after which cells were treated with K02288. Macrophages cultured with 344SQR and treated with K02288 had lower expression of p38a and p38α-regulated cytokines and chemokines compared with cells co-cultured with 344SQR only (FIGS. 3E and 3F).

In order to confirm that these findings were dependent on BMP7 secretion and not another secreted molecule, RAW 264.7 cells and mouse peritoneal macrophages were treated with BMP7 with or without its inhibitor follistatin (foll). Both RAW 264.7 cells and peritoneal macrophages had lower expression of p38a and p38α-regulated cytokines and chemokines when treated with BM7 compared to untreated control (FIG. 3G). RAW 264.7 cells had higher expression of p38a and p38α-regulated cytokines and chemokines when treated with BMP7 plus follistatin versus BMP7 only (FIG. 3H). Since 344SQR cells naturally secrete higher levels of BMP7, RAW 264.7 cells and peritoneal macrophages were co-cultured with 344SQR cells and treated with follistatin. Similarly, RAW 264.7 cells and peritoneal macrophages co-cultured with 344SQR and treated with follistatin had higher expression of p38a and p38α-regulated cytokines and chemokines versus 344SQR co-culture only (FIGS. 3I,3J). Finally, whether BMP7 regulates the expression of TNF-α, IL1-β, and CD206 in a p38a dependent manner was analyzed. To do so, the expression of p38a in RAW 264.7 cells was silenced, and then the cells were treated with BMP7. Treatment with BMP7 did not alter the expression of TNF-α, IL1-β, and CD206 in RAW 264.7 cells treated with siRNAs targeting p38a (FIG. 3K). These findings suggest that BMP7 regulates pro-inflammatory cytokine and chemokine expression via p38a in macrophages.

### Example 5—BMP7 Regulates CD4⁺ T Cell Production of IFN-γ and IL-2 Via p38α

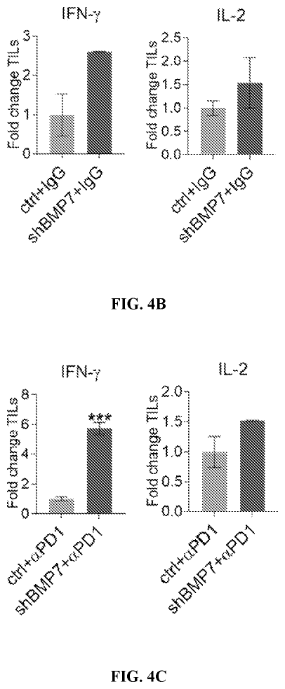

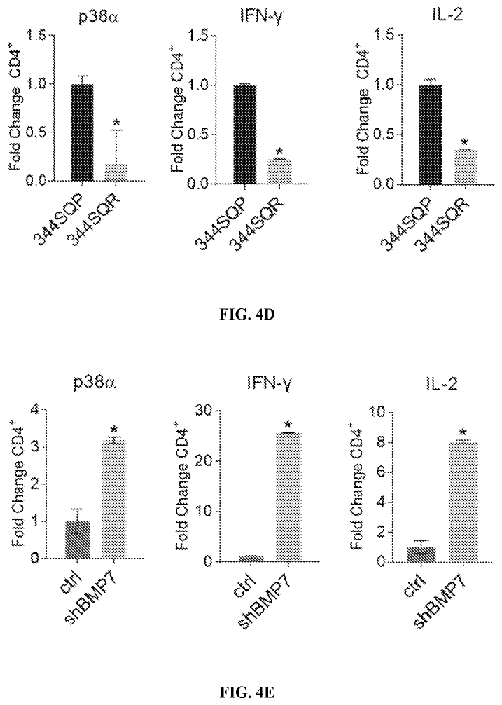

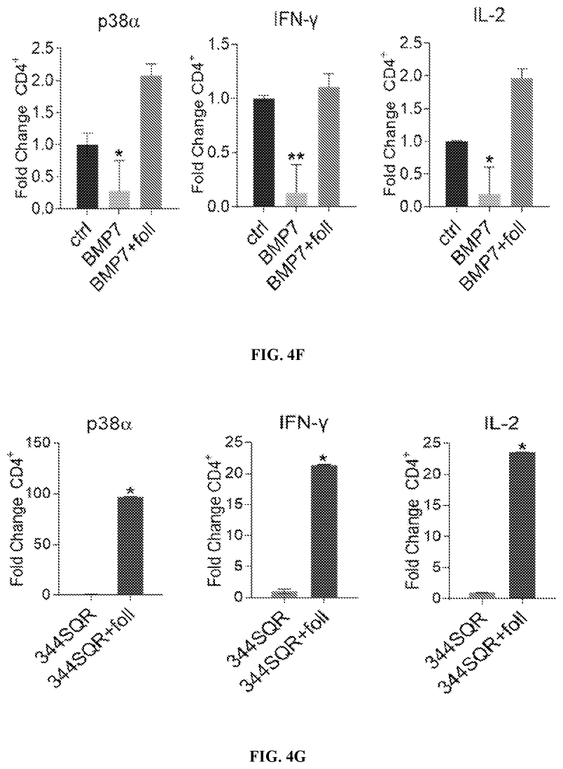

Since IFN-γ and IL-2 levels were downregulated in serum from mice bearing 344SQR tumors versus parental tumors, whether BMP7 affected the expression of IFN-γ and IL-2 in T cells via p38a was tested. Because BMP7 led to changes in p38α expression in CD4⁺ T cells but not in CD8⁺ T cells, CD4⁺ T cells were focused on here. First, to see whether BMP7 promotes p38a downregulation via SMAD1 in CD4⁺ T cells, CD4⁺ T cells were cultured and treated with BMP7 with or without follistatin for 1 hour, after which p38a expression and SMAD1/5/9 activation were evaluated. p38a expression was higher in CD4⁺ T cells treated with BMP7 plus follistatin versus BMP7 alone (FIG. 4A). On the other hand, SMAD1/5/9 activation was lower in CD4⁺ T cells treated with BMP7 plus follistatin versus BMP7 alone (FIG. 4A). Thus, BMP7 regulates p38a expression via SMAD1 signaling not only in tumors and macrophages but also in CD4⁺ T cells.

Next, IFN-γ and IL-2 expression in TILs isolated from BMP7-knockdown tumors as compared with control tumors treated with anti-PD1 was investigated. IFN-γ and IL-2 expression levels were increased in TILs from BMP7-knockdown tumors versus control (FIG. 4B). p38a was then silenced in EL4 T cells using shRNAs and IFN-γ and IL-2 expression was analyzed. As expected, silencing p38a also decreased IFN-γ and IL-2 expression in EL4 T cells (FIG. 4C).

Next, CD4⁺ T cells were co-cultured with 344SQP or 344SQR cells, and 344SQR ctrl or 344SQR shBMP7 cells, and the expression of p38α, IFN-γ, and IL-2 analyzed. For these experiments, mouse spleens were harvested, and CD4⁺ T cells collected by using magnetic beads. The collected cells were activated with CD3/CD28 antibodies before treatment. Co-culture of activated CD4⁺ T cells with 344SQR cells led to decreased p38α, IFN-γ, and IL-2 expression compared with 344SQP cells (FIG. 4D). On the other hand, co-culture of CD4⁺ T cells with 344SQR shBMP7 upregulated p38α, IFN-γ, and IL-2 expression compared with 344SQR ctrl (FIG. 4E). To confirm that these findings depended on BMP7 and not on some other secreted molecule, CD4⁺ T cells were treated with BMP7 with or without follistatin and p38α, IFN-γ, and IL-2 expression evaluated. CD4⁺ T cells had higher expression of p38α, IFN-γ, and IL-2 when treated with BM7 plus follistatin compared with BMP7 only (FIG. 4F). Because 344SQR cells naturally secrete higher levels of BMP7, CD4⁺ T cells were co-cultured with 344SQR cells and then treated with follistatin. Those CD4⁺ T cells had higher expression of p38α, IFN-γ and IL-2 versus 344SQR without follistatin (FIG. 4G). Thus, BMP7 regulates IFN-γ and IL-2 expression via p38α in CD4⁺ T cells.

### Example 6—Inhibition of BMP7 Expression Re-Sensitizes Anti-PD1-Resistant Tumors

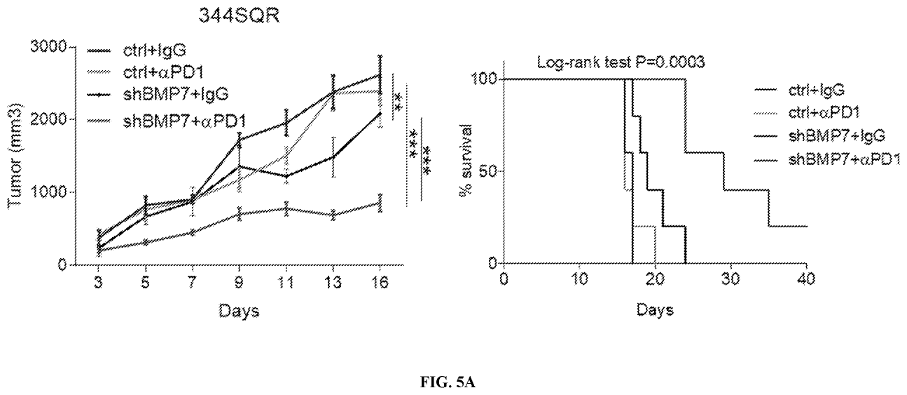

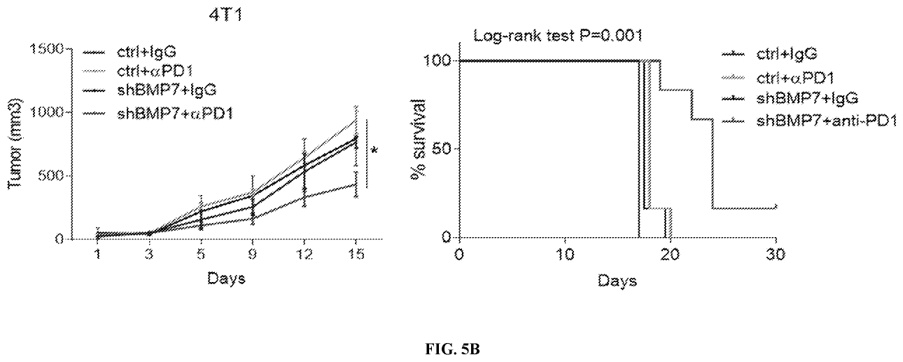

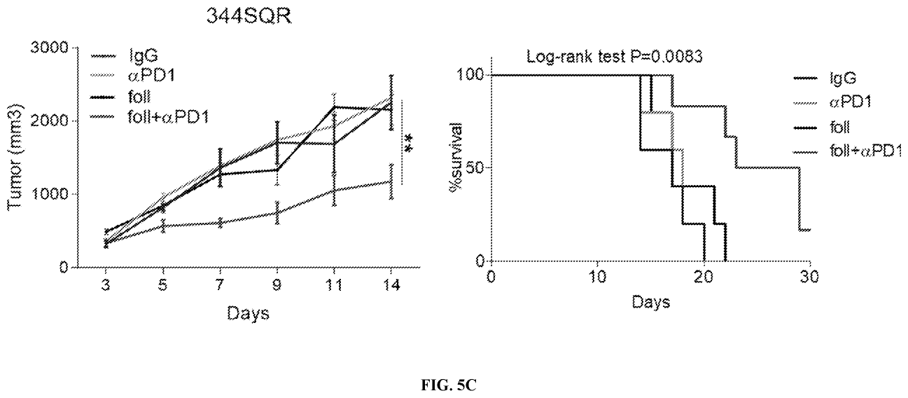

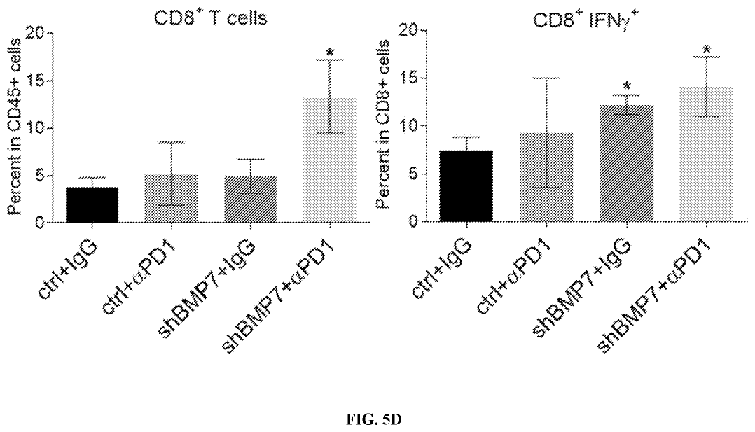

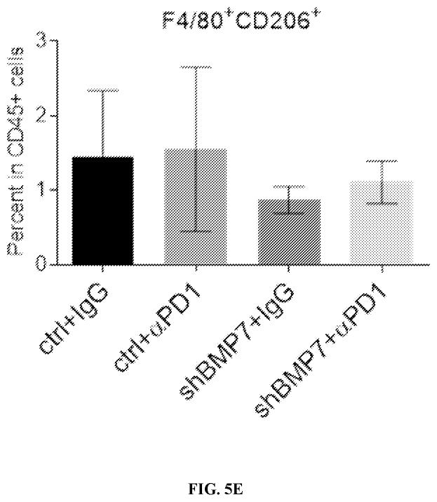

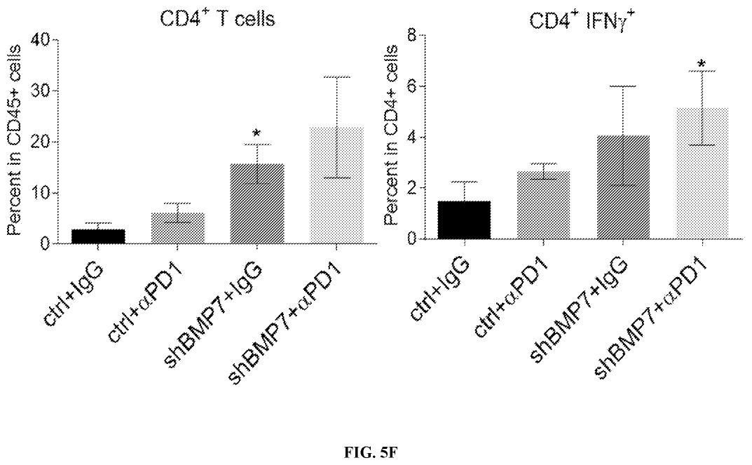

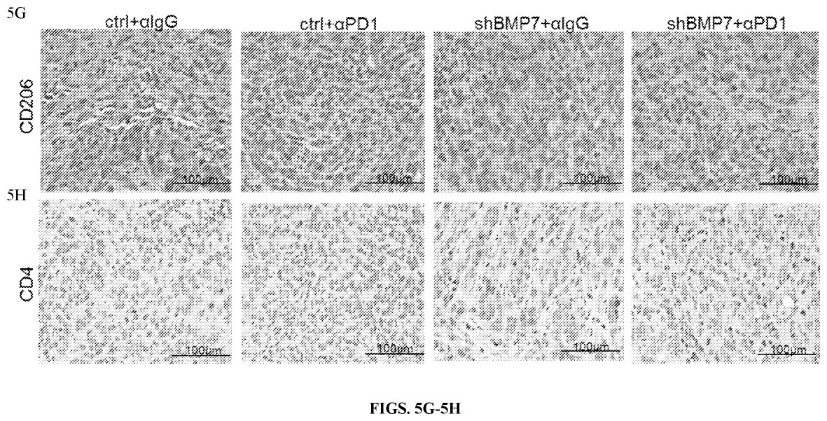

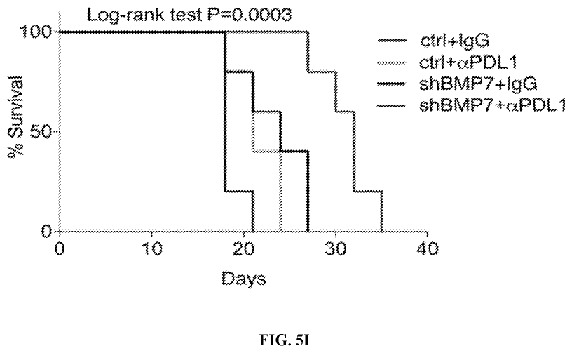

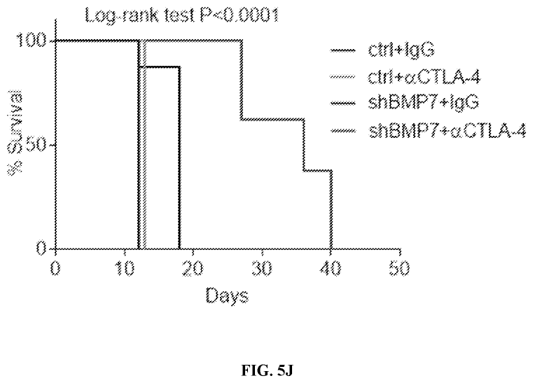

Next, whether BMP7 knockdown could sensitize anti-PD1-resistant tumors to immunotherapy was tested in two different in vivo models that are resistant to immunotherapies 344SQR and triple negative breast cancer 4T1 model. 344SQR ctrl and 344SQR shBMP7 cells were injected into 129Sv/Ev mice and the mice were treated with IgG control or anti-PD1 (FIG. 5A). 4T1 ctrl or 4T1 shBMP7 cells were injected into BALB/c mice and the mice were treated with IgG control or anti-PD1 (FIG. 5B). BMP7 knockdown was found to re-sensitize tumors to anti-PD1 and extended mouse survival relative to the control group. Whether BMP7 inhibition via follistatin could re-sensitize resistant tumors was evaluated. BMP7 inhibition via follistatin decreased tumor growth and extended survival compared with anti-PD1 therapy only (FIG. 5C). Increased percentages and activation of CD8⁺ T cells only were found in the BMP7-knockdown tumors treated with anti-PD1 versus BMP7-knockdown tumors treated with IgG or control tumors treated with IgG or anti-PD1 (FIG. 5D). Next, the percentages of M2 macrophages (CD206 marker) were evaluated in BMP7-knockdown tumors treated with anti-PD1 (versus IgG-treated control), and BMP7-knockdown tumors treated with IgG or anti-PD1 had decreased percentages of M2 macrophages compared with control tumors treated with IgG or anti-PD1 (FIG. 5E). Then, percentages and activation of CD4⁺ T cells (via IFN-γ production) in BMP7-knockdown tumors treated with anti-PD1 (compared with IgG-treated controls) were evaluated. The percentage of CD4⁺ T cells in BMP7-knockdown tumors treated with anti-PD1 or IgG increased compared with control tumors treated with IgG or anti-PD1 (FIG. 5F). Also, the number of CD4⁺ IFN-γ⁺ T cells in BMP7-knockdown tumors treated with anti-PD1 or IgG was higher than in control tumors treated with IgG (FIG. 5F). M2 macrophage and CD4⁺ T cell infiltration was evaluated by IHC staining. Concordant with the flow cytometry data, infiltration of M2 macrophages was decreased in BMP7-knockdown tumors treated with IgG or anti-PD1 compared with control tumors (FIG. 5G). On the other hand, infiltration of CD4⁺ T cells was higher in BMP7-knockdown tumors treated with IgG or anti-PD1 compared with control tumors (FIG. 5H). The combination of BMP7 knockdown and anti-CTLA4 or anti-PDL1 was also tested. Antibodies to both PDL1 and CTLA4 increased survival in combination with BMP7-knockdown compared with control (FIGS. 5I,5J). Collectively, BMP7 inhibition or treatment with follistatin may represent a new therapeutic approach to overcome resistance to immunotherapies such as anti-PD1, anti-CTLA4, and anti-PDL1.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Alarmo et al., “A comprehensive expression survey of bone morphogenetic proteins in breast cancer highlights the importance of BMP4 and BMP7,” Breast Cancer Research and Treatment, 103:239-246 (2007).
Alarmo et al., “Bone morphogenetic protein 7 is widely overexpressed in primary breast cancer,” Genes, Chromosomes & Cancer, 45:411-419 (2006).
Amirouche et al., “Activation of p38 signaling increases utrophin A expression in skeletal muscle via the RNA-binding protein KSRP and inhibition of AU-rich element-mediated mRNA decay: implications for novel DMD therapeutics,” Hum. Mol. Genet., 22:3093-3111 (2013).
Aoki et al., “Expression of BMP-7 in human gastric cancer and its clinical significance,” British Journal of Cancer, 104:714-718 (2011).
Awazu et al., “BMP7 dose-dependently stimulates proliferation and cadherin-11 expression via ERK and p38 in a murine metanephric mesenchymal cell line,” Physiological Reports, 5:e13378 (2017).
Baldassare et al., “The role of p38 mitogen-activated protein kinase in IL-1 beta transcription,” Journal of Immunology, 162:5367-5373 (1999).
Brahmer et al., “Safety and activity of anti-PD-L1 antibody in patients with advanced cancer,” N. Engl. J. Med., 366:2455-2465 (2012).
Byeon et al., “p38-targeted inhibition of interleukin-12 expression by ethanol extract from Cordyceps bassiana in lipopolysaccharide-activated macrophages,” Immunopharmacol. Immunotoxicol., 33:90-96 (2011).
Cancer Genome Atlas Research N. Comprehensive molecular profiling of lung adenocarcinoma. Nature 2014; 511(7511):543-50.
Chen et al., “Expression of bone morphogenetic protein 7 in lung cancer and its biological impact on lung cancer cells,” Anticancer Research. 30:1113-1120 (2010).
Chen & Ten Dijke, “Immunoregulation by members of the TGFbeta superfamily,” Nature Reviews Immunology, 16:723-740 (2016).
Cortez et al., “Therapeutic delivery of miR-200c enhances radiosensitivity in lung cancer,” Molecular Therapy, 22:1494-1503 (2014).
Crawley et al., “T cell proliferation in response to interleukins 2 and 7 requires p38MAP kinase activation,” J. Biol. Chem., 272:15023-15027 (1997).
DeSilva et al., “The p38 mitogen-activated protein kinase pathway in activated and anergic Th1 cells,” Cell Immunol., 180:116-123 (1997).
Dunn & Rao, “Epigenetics and immunotherapy: The current state of play,” Mol. Immunol., 87:227-239 (2017).
Garcia et al., “A Mycoplasma fermentans-derived synthetic lipopeptide induces AP-1 and NF-kappaB activity and cytokine secretion in macrophages via the activation of mitogen-activated protein kinase pathways,” J. Biol. Chem., 273:34391-34398 (1998).
Gavenis et al., “Effects of controlled released BMP-7 on markers of inflammation and degradation during the cultivation of human osteoarthritic chondrocytes,” J. Biomater. Appl., 26:419-433 (2011).
Gettinger & Herbst, “B7-H1/PD-1 blockade therapy in non-small cell lung cancer: current status and future direction,” Cancer J., 20:281-289 (2014).
Gould et al., “BMP-7 regulates chemokine, cytokine, and hemodynamic gene expression in proximal tubule cells,” Kidney Int., 61:51-60 (2002).
Hong et al., “Effect of bone morphogenetic protein-6 on macrophages,” Immunology, 128:e442-e450 (2009).
Hu et al., “p38MAPK acts in the BMP7-dependent stimulatory pathway during epithelial cell morphogenesis and is regulated by Smad1,”J. Biol. Chem., 279:12051-12059 (2004).
Iwasaki et al., “Specific activation of the p38 mitogen-activated protein kinase signaling pathway and induction of neurite outgrowth in PC12 cells by bone morphogenetic protein-2,” J. Biol. Chem., 274:26503-26510 (1999).
Kelderman et al., “Acquired and intrinsic resistance in cancer immunotherapy,” Mol. Oncol., 8:1132-1139 (2014).
Kim et al., “Inhibition of p38 and ERK MAP kinases blocks endotoxin-induced nitric oxide production and differentially modulates cytokine expression,” Pharmacological Research, 49:433-439 (2004).
Kogkopoulou et al., “Conditional up-regulation of IL-2 production by p38 MAPK inactivation is mediated by increased Erk1/2 activity,” J. Leukoc. Biol., 79:1052-1060, (2006).
Kretzschmar et al., “The TGF-beta family mediator Smad1 is phosphorylated directly and activated functionally by the BMP receptor kinase,” Genes & Development, 11:984-995 (1997a).
Kretzschmar et al., “Opposing BMP and EGF signalling pathways converge on the TGF-beta family mediator Smad1,” Nature, 389:618-622 (1997b).
Kroeger et al., “Aberrant CpG island methylation in acute myeloid leukemia is accentuated at relapse,” Blood, 112:1366-1373 (2008).
Kron et al., “Discovery of novel hypermethylated genes in prostate cancer using genomic CpG island microarrays,”PLoS One, 4:e4830 (2009).
Kwon et al., “Bone morphogenetic protein-6 induces the expression of inducible nitric oxide synthase in macrophages,” Immunology, 128:e758-e765 (2009).
Lee et al., “A protein kinase involved in the regulation of inflammatory cytokine biosynthesis,” Nature, 372:739-746 (1994).
Lee et al., “BMP-6 in renal cell carcinoma promotes tumor proliferation through IL-1β-dependent M2 polarization of tumor-associated macrophages,” Cancer Res., 73:3604-3614 (2013).
Lee et al., “Both the Smad and p38 MAPK pathways play a crucial role in Runx2 expression following induction by transforming growth factor-beta and bone morphogenetic protein,” Oncogene, 21:7156-7163 (2002).
Li et al., “BMP7 reduces inflammation and oxidative stress in diabetic tubulopathy,” Clin. Sci. (Lond.), 128:269-280 (2015).
Li et al., “TCPA: a resource for cancer functional proteomics data,” Nat. Methods, 10:1046-1047 (2013).
Liu et al., “A human Mad protein acting as a BMP-regulated transcriptional activator,” Nature, 381:620-623 (1996).
Liu et al., “Molecular impact of bone morphogenetic protein 7, on lung cancer cells and its clinical significance,” International Journal of Molecular Medicine, 29:1016-1024 (2012).
Loeser et al., “Methylation of the OP-1 promoter: potential role in the age-related decline in OP-1 expression in cartilage,” Osteoarthritis Cartilage, 17:513-517 (2009).
Malhotra & Kang, “SMAD regulatory networks construct a balanced immune system,” Immunology, 139:1-10 (2013).
Martinez et al., “The canonical BMP signaling pathway is involved in human monocyte-derived dendritic cell maturation,” Immunology and Cell Biology, 89:610-618 (2011).
Martinez et al., “The BMP Pathway Participates in Human Naive CD4+ T Cell Activation and Homeostasis,” PLoS One, 10:e0131453 (2015).
Matsuda et al., “T lymphocyte activation signals for interleukin-2 production involve activation of MKK6-p38 and MKK7-SAPK/JNK signaling pathways sensitive to cyclosporin A,” J. Biol. Chem., 273:12378-12382 (1998).
Mavropoulos et al., “Stabilization of IFN-gamma mRNA by MAPK p38 in IL-12- and IL-18-stimulated human NK cells,” Blood, 105:282-288 (2005).
Megumi et al., “Clinicopathological significance of BMP7 expression in esophageal squamous cell carcinoma,” Annals of Surgical Oncology, 19:2066-2071 (2012).
Motoyama et al., “Clinical significance of BMP7 in human colorectal cancer,” Annals of Surgical Oncology, 15:1530-1537 (2008).
Nguyen et al., “IL-12 enhances IL-2 function by inducing CD25 expression through a p38 mitogen-activated protein kinase pathway,” Eur. J. Immunol., 30:1445-1452 (2000).
Owens et al., “Inhibition of BMP signaling suppresses metastasis in mammary cancer,” Oncogene, 34:2437-2449 (2015).
Rincon et al., “Interferon-gamma expression by Th1 effector T cells mediated by the p38 MAP kinase signaling pathway,” EMBO J., 17:2817-2829 (1998).
Rocher et al., “Bone morphogenetic protein 7 polarizes THP-1 cells into M2 macrophages,” Canadian Journal of Physiology and Pharmacology, 90:947-951 (2012).
Rocher & Singla, “SMAD-PI3K-Akt-mTOR pathway mediates BMP-7 polarization of monocytes into M2 macrophages,” PLoS ONE, 8:e84009 (2013).
Rothhammer et al., “Bone morphogenic proteins are overexpressed in malignant melanoma and promote cell invasion and migration,” Cancer Res., 65:448-456 (2005).
Shoulders et al., “Macrophage depletion by clodronate attenuates bone morphogenetic protein-7 induced M2 macrophage differentiation and improved systolic blood velocity in atherosclerosis,” Transl. Res., 203:1-14 (2019).
Singla et al., “BMP-7 Treatment Increases M2 Macrophage Differentiation and Reduces Inflammation and Plaque Formation in Apo E−/− Mice,” PLoS ONE, 11:e0147897 (2016).
Takahashi et al., “Bone morphogenetic protein 6 (BMP6) and BMP7 inhibit estrogen-induced proliferation of breast cancer cells by suppressing p38 mitogen-activated protein kinase activation,” The Journal of Endocrinology, 199:445-455 (2008).
Takimoto et al., “Smad2 and Smad3 are redundantly essential for the TGF-beta-mediated regulation of regulatory T plasticity and Th1 development,” J. Immunol., 185:842-855 (2010).
Topalian et al., “Targeting the PD-1/B7-H1(PD-L1) pathway to activate anti-tumor immunity,” Curr. Opin. Immunol., 24:207-212 (2012a).
Topalian et al., “Safety, activity, and immune correlates of anti-PD-1 antibody in cancer,” N. Engl. J. Med., 366:2443-2454 (2012b).
Valledor et al., “Selective roles of MAPKs during the macrophage response to IFN-gamma,” Journal of Immunology, 180:4523-4529 (2008).
Veiopoulou et al., “IL-2 and IL-10 production by human CD4+ T cells is differentially regulated by p38: mode of stimulation-dependent regulation of IL-2,” Neuroimmunomodulation, 11:199-208 (2004).
Wagner & Nebreda, “Signal integration by JNK and p38 MAPK pathways in cancer development,” Nat. Rev. Cancer, 9:537-549 (2009).
Wang et al., “Bone morphogenetic protein-7 prevented epithelial-mesenchymal transition in RLE-6TN cells,” Toxicology Research, 5:931-937 (2016).
Wang et al., “Suppression of type I IFN signaling in tumors mediates resistance to anti-PD-1 treatment that can be overcome by radiotherapy,” Cancer Res., 77:839-850 (2016).
Yang et al., “Functional roles of p38 mitogen-activated protein kinase in macrophage-mediated inflammatory responses,”Mediators Inflamm., 2014:352371 (2014).
Yang et al., “IL-18-stimulated GADD45 beta required in cytokine-induced, but not TCR-induced, IFN-gamma production,” Nature Immunology, 2:157-164 (2001).
Yang et al., “LPS converts Gr-1(+)CD115(+) myeloid-derived suppressor cells from M2 to M1 via P38 MAPK,” Experimental Cell Research, 319:1774-1783 (2013).
Yang et al., “Nanostructured, self-assembling peptide K5 blocks TNF-alpha and PGE(2) production by suppression of the AP-1/p38 pathway,” Mediators Inflamm., 2012:489810 (2012).
Yu et al., “Regulation and phenotype of an innate Th1 cell: role of cytokines and the p38 kinase pathway,” Journal of Immunology, 171:6112-6118 (2003).
Zhang et al., “p38 mitogen-activated protein kinase mediates signal integration of TCR/CD28 costimulation in primary murine T cells,” Journal of Immunology, 162:3819-3829 (1999).
Zhu et al., “Regulation of TNF expression by multiple mitogen-activated protein kinase pathways,” Journal of Immunology, 164:6349-6358 (2000).

1. A method for the treatment of a cancer in a patient, the method comprising administering to the patient a combined effective amount of a BMP7 inhibitor and an immune checkpoint inhibitor.

2. The method of claim 1, wherein the patient has previously failed to respond to the administration of an immune checkpoint inhibitor.

3. The method of claim 2, wherein the immune checkpoint inhibitor comprises an anti-PD1, anti-PD-L1 therapy, and/or anti-CTLA-4 therapy.

4. The method of claim 1, wherein the patient's cancer expresses an increased level of BMP7 relative to a BMP7 level in a reference sample.

5. The method of claim 1, wherein the patient's serum comprises an increased level of BMP7 relative to a BMP7 level in a reference sample.

6. The method of claim 1, wherein the patient's cancer expresses an increased level of beta-catenin, Sox2, and/or PARP1 relative to a beta-catenin, Sox2, and/or PARP1 level in a reference sample.

7. The method of claim 1, wherein the patient's cancer expresses a decreased level of CDKN2A, p38α, PTEN, PD-L1, YAP1_pS127, and/or granzyme B relative to a CDKN2A, p38α, PTEN, PD-L1, YAP1_pS127, and/or granzyme B level in a reference sample.

8. The method of claim 1, wherein a tumor infiltrating lymphocyte in the patient's cancer expresses a decreased level of IL-1α, TNF-α, IFN-γ, and/or IL-2 relative to an IL-1α, TNF-α, IFN-γ, and/or IL-2 level in a reference sample.

9. The method of any one of claims 4 and 6-8, wherein the reference sample is sourced from healthy or non-cancerous tissue from the patient.

10. The method of any one of claims 4-8, wherein the reference sample is sourced from a healthy subject.

11. The method of claim 1, wherein the BMP7 inhibitor comprises a BMP7 antagonist protein, a BMP7 neutralizing antibody, an inhibitory nucleic acid targeting BMP7 mRNA, or a BMP7 antagonist small molecule.

12. The method of claim 11, wherein the BMP7 antagonist protein is follistatin or uterine sensitization-associated gene-1 (USAG-1).

13. The method of claim 11, wherein the BMP7 antagonist protein is PEGylated.

14. The method of claim 11, wherein the BMP7 antagonist small molecule is K02288.

15. The method of claim 11, wherein the inhibitory nucleic acid targeting BMP7 mRNA comprises a BMP7 shRNA or siRNA.

16. The method of claim 1, wherein the BMP7 inhibitor is comprised in a lipid nanoparticle.

17. The method of claim 16, wherein the lipid nanoparticle is an exosome.

18. The method of claim 1, wherein the BMP7 inhibitor is comprised in a nanoshuttle for controlled intratumoral delivery.

19. The method of any one of claims 1-18, wherein the immune checkpoint inhibitor comprises one or more of an anti-PD1 therapy, an anti-PD-L1 therapy, and an anti-CTLA-4 therapy.

20. The method of claim 19, wherein the anti-PD1 therapy comprises nivolumab, pembrolizumab, pidilizumab, AMP-223, AMP-514, cemiplimab, or PDR-001.

21. The method of claim 19, wherein the anti-PD-L1 therapy comprises atezolizumab, avelumab, durvalumab, BMS-036559, or CK-301.

22. The method of claim 19, wherein the anti-CTLA-4 therapy comprises ipilimumab or tremelimumab.

23. The method of any one of claims 1-22, further comprising administering a further anti-cancer therapy to the patient.

24. The method of claim 23, wherein the second anti-cancer therapy is a surgical therapy, chemotherapy, radiation therapy, cryotherapy, hormonal therapy, toxin therapy, immunotherapy, or cytokine therapy.

25. The method of claim 24, wherein the surgical therapy comprises a pneumonectomy, a lobectomy, a segmentectomy, a wedge resection, or a sleeve resection.

26. The method of claim 24, wherein the radiation therapy comprises external beam radiation therapy or brachytherapy.

27. The method of claim 24, wherein the chemotherapy comprises the administration of one or more agents selected from the group consisting of cisplatin, carboplatin, paclitaxel, albumin-bound paclitaxel, docetaxel, gemcitabine, vinorelbine, irinotecan, etoposide, vinblastine, and pemetrexed.

28. The method of claim 23, wherein the further anti-cancer therapy comprises erlotinib, afatinib, gefitinib, osimertinib, or dacomitinib if the patient's cancer expresses an increased level of EGFR relative to a reference level.

29. The method of claim 23, wherein the further anti-cancer therapy comprises crizotinib, ceritinib, alectinib, brigatinib, or lorlatinib if the patient's cancer has an ALK gene rearrangement.

30. The method of claim 23, wherein the further anti-cancer therapy comprises dabrafenib or trametinib if the patient's cancer expresses an altered BRAF protein.

31. The method of any one of claims 1-30, wherein the cancer is a lung cancer or a breast cancer.

32. The method of claim 31, wherein the lung cancer is a non-small cell lung cancer.

33. The method of claim 31, wherein the breast cancer is triple-negative breast cancer.

34. The method of any one of claims 1-33, wherein the patient has previously undergone at least one round of anti-cancer therapy.

35. The method of any one of claims 1-34, wherein the patient is a human.

36. The method of any one of claims 3-7, further comprising reporting the BMP7, beta-catenin, Sox2, PARP1, CDKN2A, p38α, PTEN, PD-L1, YAP1_pS127, granzyme B, IL-1α, TNF-α, IFN-γ, and/or IL-2 expression level.

37. The method of claim 36, wherein the reporting comprises preparing a written or electronic report.

38. The method of claim 37, further comprising providing the report to the subject, a doctor, a hospital, or an insurance company.

39. A method of selecting a patient having a cancer for treatment with a combined effective amount of a BMP7 inhibitor and an immune checkpoint inhibitor, the method comprising (a) determining whether the patient's cancer has an increased level of BMP7 relative to a BMP7 level in a reference sample, and (b) selecting the patient for treatment if the patient's cancer has an increased level of BMP7 relative to a BMP7 level in a reference sample.

40. The method of claim 39, further comprising administering a combined effective amount of a BMP7 inhibitor and an immune checkpoint inhibitor to the selected patient.

41. The method of claim 39, further comprising selecting the patient for treatment if the patient has previously failed to respond to the administration of an immune checkpoint inhibitor.

42. The method of claim 41, wherein the immune checkpoint inhibitor comprises an anti-PD1 and/or anti-PD-L1 therapy.

43. The method of claim 39, further comprising selecting the patient for treatment if the patient's serum comprises an increased level of BMP7 relative to a BMP7 level in a reference sample.

44. The method of claim 39, further comprising selecting the patient for treatment if the patient's cancer expresses an increased level of beta-catenin, Sox2, and/or PARP1 relative to a beta-catenin, Sox2, and/or PARP1 level in a reference sample.

45. The method of claim 39, further comprising selecting the patient for treatment if the patient's cancer expresses a decreased level of CDKN2A, p38α, PTEN, PD-L1, YAP1_pS127, and/or granzyme B relative to a CDKN2A, p38α, PTEN, PD-L1, YAP1_pS127, and/or granzyme B level in a reference sample.

46. The method of claim 39, further comprising selecting the patient for treatment if a tumor infiltrating lymphocyte in the patient's cancer expresses a decreased level of IL-1α, TNF-α, IFN-γ, and/or IL-2 relative to an IL-1α, TNF-α, IFN-γ, and/or IL-2 level in a reference sample.

47. The method of any one of claims 39 and 44-46, wherein the reference sample is sourced from healthy or non-cancerous tissue from the patient.

48. The method of any one of claims 39 and 43-46, wherein the reference sample is sourced from a healthy subject.

49. The method of any one of claims 39-48, wherein the BMP7 inhibitor comprises a BMP7 antagonist protein, a BMP7 neutralizing antibody, an inhibitory nucleic acid targeting BMP7 mRNA, or a BMP7 antagonist small molecule.

50. The method of claim 49, wherein the BMP7 antagonist protein is follistatin or uterine sensitization-associated gene-1 (USAG-1).

51. The method of claim 49, wherein the BMP7 antagonist protein is PEGylated.

52. The method of claim 49, wherein the BMP7 antagonist small molecule is K02288.

53. The method of claim 49, wherein the inhibitory nucleic acid targeting BMP7 mRNA comprises a BMP7 shRNA or siRNA.

54. The method of any one of claims 39-48, wherein the BMP7 inhibitor is comprised in a lipid nanoparticle.

55. The method of claim 54, wherein the lipid nanoparticle is an exosome.

56. The method of any one of claims 40-48, wherein the BMP7 inhibitor is comprised in a nanoshuttle for controlled intratumoral delivery.

57. The method of any one of claims 39-48, wherein the immune checkpoint inhibitor comprises one or more of an anti-PD1 therapy, an anti-PD-L1 therapy, and an anti-CTLA-4 therapy.

58. The method of claim 57, wherein the anti-PD1 therapy comprises nivolumab, pembrolizumab, pidilizumab, AMP-223, AMP-514, cemiplimab, or PDR-001.

59. The method of claim 57, wherein the anti-PD-L1 therapy comprises atezolizumab, avelumab, durvalumab, BMS-036559, or CK-301.

60. The method of claim 57, wherein the anti-CTLA-4 therapy comprises ipilimumab or tremelimumab.

61. The method of claim 40, further comprising administering a further anti-cancer therapy to the patient.

62. The method of claim 61, wherein the second anti-cancer therapy is a surgical therapy, chemotherapy, radiation therapy, cryotherapy, hormonal therapy, toxin therapy, immunotherapy, or cytokine therapy.

63. The method of claim 62, wherein the surgical therapy comprises a pneumonectomy, a lobectomy, a segmentectomy, a wedge resection, or a sleeve resection.

64. The method of claim 62, wherein the radiation therapy comprises external beam radiation therapy or brachytherapy.

65. The method of claim 62, wherein the chemotherapy comprises the administration of one or more agents selected from the group consisting of cisplatin, carboplatin, paclitaxel, albumin-bound paclitaxel, docetaxel, gemcitabine, vinorelbine, irinotecan, etoposide, vinblastine, and pemetrexed.

66. The method of claim 61, wherein the further anti-cancer therapy comprises erlotinib, afatinib, gefitinib, osimertinib, or dacomitinib if the patient's cancer expresses an increased level of EGFR relative to a reference level.

67. The method of claim 61, wherein the further anti-cancer therapy comprises crizotinib, ceritinib, alectinib, brigatinib, or lorlatinib if the patient's cancer has an ALK gene rearrangement.

68. The method of claim 61, wherein the further anti-cancer therapy comprises dabrafenib or trametinib if the patient's cancer expresses an altered BRAF protein.

69. The method of any one of claims 39-68, wherein the cancer is a lung cancer or a breast cancer.

70. The method of claim 69, wherein the lung cancer is a non-small cell lung cancer.

71. The method of claim 69, wherein the breast cancer is a triple-negative breast cancer.

72. The method of any one of claims 39-71, wherein the patient has previously undergone at least one round of anti-cancer therapy.

73. The method of any one of claims 39-72, wherein the patient is a human.

74. The method of claim 39, further comprising reporting the BMP7 level in the patient's cancer.

75. The method of claim 74, wherein the reporting comprises preparing a written or electronic report.

76. The method of claim 75, further comprising providing the report to the subject, a doctor, a hospital, or an insurance company.

77. A pharmaceutical formulation comprising a BMP7 inhibitor and an immune checkpoint inhibitor.

78. The formulation of claim 77, wherein the BMP7 inhibitor comprises a BMP7 antagonist protein, a BMP7 neutralizing antibody, an inhibitory nucleic acid targeting BMP7 mRNA, or a BMP7 antagonist small molecule.

79. The formulation of claim 78, wherein the BMP7 antagonist protein is follistatin or uterine sensitization-associated gene-1 (USAG-1).

80. The method of claim 78, wherein the BMP7 antagonist protein is PEGylated.

81. The formulation of claim 78, wherein the BMP7 antagonist small molecule is K02288.

82. The formulation of claim 78, wherein the inhibitory nucleic acid targeting BMP7 mRNA comprises a BMP7 shRNA or siRNA.

83. The formulation of claim 77, wherein the BMP7 inhibitor is comprised in a lipid nanoparticle.

84. The formulation of claim 83, wherein the lipid nanoparticle is an exosome.

85. The formulation of any one of claims 77-85, wherein the BMP7 inhibitor is comprised in a nanoshuttle for controlled intratumoral delivery.

86. The formulation of any one of claims 77-85, wherein the immune checkpoint inhibitor comprises one or more of an anti-PD1 therapy, an anti-PD-L1 therapy, and an anti-CTLA-4 therapy.

87. The formulation of claim 86, wherein the anti-PD1 therapy comprises nivolumab, pembrolizumab, pidilizumab, AMP-223, AMP-514, cemiplimab, or PDR-001.

88. The formulation of claim 86, wherein the anti-PD-L1 therapy comprises atezolizumab, avelumab, durvalumab, BMS-036559, or CK-301.

89. The formulation of claim 86, wherein the anti-CTLA-4 therapy comprises ipilimumab or tremelimumab.

90. Use of a pharmaceutical formulation of any one of claims 77-89 in the manufacture of a medicament for treating a cancer in a subject.

91. A pharmaceutical formulation of any one of claims 77-89 for use in treating a cancer in a subject.
